(19) United States
(12) Patent Application Publication
(10) Pub. No.: US 2023/0103089 A1
(43) Pub. Date: Mar. 30, 2023

(54) RESIST UNDERLAYER COMPOSITION AND METHOD OF FORMING PATTERNS USING THE COMPOSITION

(71) Applicant: SAMSUNG SDI CO., LTD., Yongin-si (KR)

(72) Inventors: Yoojeong CHOI, Suwon-si (KR); Soonhyung KWON, Suwon-si (KR); Minsoo KIM, Suwon-si (KR); Seongjin KIM, Suwon-si (KR); Jaeyeol BAEK, Suwon-si (KR); Hwayoung JIN, Suwon-si (KR); Ran NAMGUNG, Suwon-si (KR); Hyeon PARK, Suwon-si (KR); Daeseok SONG, Suwon-si (KR)

(21) Appl. No.: 17/868,563

(22) Filed: Jul. 19, 2022

(30) Foreign Application Priority Data
Jul. 30, 2021 (KR) 10-2021-0100893

(51) Int. Cl. G03F 7/11; C07D 251/32; C07D 405/06; C07D 209/86; C07D 209/62; C07D 333/54; C07D 339/08; C07C 39/23; C08K 5/378; C08K 5/3417; C08K 5/45

(52) U.S. Cl. CPC G03F 7/11; C07D 251/32; C07D 405/06; C07D 209/86; C07D 209/62; C07D 333/54; C07D 339/08; C07C 39/23; C08K 5/378; C08K 5/3417; C08K 5/45

(57) ABSTRACT

A resist underlayer composition including a polymer including a main chain, a side chain, or a main chain and a side chain including a heterocycle including two or more nitrogen atoms in the ring of the heterocycle, a compound including a moiety represented by Chemical Formula 1, and a solvent is provided. A method of forming patterns using the resist underlayer composition is also provided

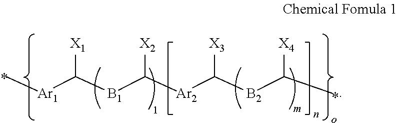

### CROSS-REFERENCE TO RELATED APPLICATION

This application claims priority to and the benefit of Korean Patent Application No. 10-2021-0100893, filed in the Korean Intellectual Property Office on Jul. 30, 2021, the entire content of which is incorporated hereby by reference.

### BACKGROUND

### 1. Field

Aspects of one or more embodiments of the present disclosure relate to a resist underlayer composition, and a method of forming patterns utilizing the same.

### 2. Description of the Related Art

Recently, the semiconductor industry has developed to an ultra-fine technique having a pattern in the nanometer scale/size (e.g., several to several tens of nanometers). Such ultra-fine technique essentially needs (requires) effective lithographic techniques.

The lithographic technique is a processing method including coating a photoresist layer on a semiconductor substrate such as a silicon wafer to form a thin layer, irradiating with activating radiation such as ultraviolet rays through a mask pattern on which the device pattern is drawn and then developing the resultant to obtain a photoresist pattern, and etching the substrate utilizing the photoresist pattern as a protective layer to form a fine pattern corresponding to the pattern, on the surface of the substrate.

In order to realize ultra-fine technology, a patterning material suitable for it is required or desired, and research on it is being actively conducted. For fine patterning, it is required or desired that the resist underlayer should have a good or suitable close-contacting property between the underlayer and the resist, so that resist pattern collapse does not occur even in fine patterning, an exposure time should be shortened in the etching process by having a faster etch rate than resist or by applying as thin as possible, and sensitivity to the exposure light source should be improved to improve patterning performance, and efficiency for the light source should be high.

### SUMMARY

An aspect of one or more embodiments of the present disclosure is directed toward a resist underlayer composition in which the pattern collapse of the resist is not caused even in the fine patterning process, an etching process time may be shortened because it is formed into an ultra-thin layer, and coating uniformity, gap-fill characteristics, and resist pattern-forming capability may be improved by improving the crosslinking property.

Another aspect of one or more embodiments of the present disclosure is directed toward a method of forming patterns utilizing the resist underlayer composition.

Additional aspects will be set forth in part in the description which follows and, in part, will be apparent from the description, or may be learned by practice of the presented embodiments of the disclosure.

A resist underlayer composition according to an embodiment includes a polymer including a main chain, a side chain, or a main chain and a side chain including a heterocycle including two or more nitrogen atoms in the ring, a compound including a moiety represented by Chemical Formula 1, and a solvent.

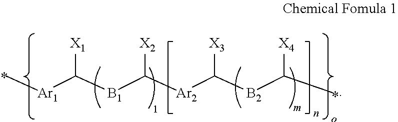

In Chemical Formula 1,

Ar₁ may be a group including a heterocycle,

Ar₂ may be a substituted or unsubstituted C6 to C30 aromatic hydrocarbon group,

B₁ and B₂ may each independently be a single bond, a substituted or unsubstituted C1 to C10 aliphatic hydrocarbon group, a substituted or unsubstituted C6 to C30 aromatic hydrocarbon group, or one or more combinations thereof,

X₁ to X₄ may each independently be hydrogen, deuterium, a hydroxy group, a thiol group, a cyano group, a substituted or unsubstituted amino group, a halogen atom, a substituted or unsubstituted C1 to C30 aliphatic hydrocarbon group, a substituted or unsubstituted C1 to C30 alkoxy group, a substituted or unsubstituted C6 to C30 aromatic hydrocarbon group, a substituted or unsubstituted C2 to C30 heterocyclic group, or one or more combinations thereof,

I, m, and n may each independently be an integer from 0 to 5, and

o may be an integer from 1 to 30.

The polymer may include any one or more of the structural units represented by Chemical Formula 2 to Chemical Formula 5.

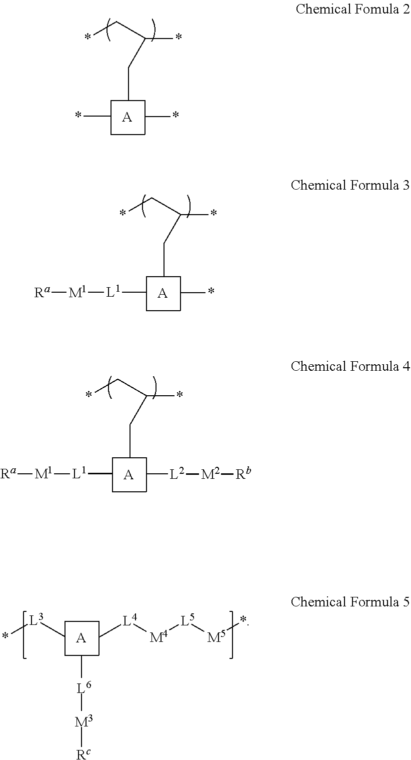

In Chemical Formula 2 to Chemical Formula 5,

A may be a heterocycle containing two or more nitrogen atoms in the ring,

R^{a}, R^{b}, and R^{c} may each independently be a hydroxy group, a substituted or unsubstituted C1 to C10 alkyl group, a substituted or unsubstituted C2 to C10 alkenyl group, a substituted or unsubstituted C2 to C10 alkynyl group, a substituted or unsubstituted C1 to C10 heteroalkyl group, a substituted or unsubstituted C1 to C10 heteroalkenyl group, a substituted or unsubstituted C1 to C10 heteroalkynyl group, a substituted or unsubstituted C3 to C20 cycloalkyl group, a substituted or unsubstituted C2 to C20 heterocycloalkyl group, a substituted or unsubstituted C6 to C20 aryl group, a substituted or unsubstituted C1 to C20 heteroaryl group, or one or more combinations thereof,

L¹ to L⁵ may each independently be a single bond, a substituted or unsubstituted C1 to C10 alkylene group, a substituted or unsubstituted C1 to C10 heteroalkylene group, a substituted or unsubstituted C3 to C20 cycloalkylene group, a substituted or unsubstituted C2 to C20 heterocycloalkylene group, a substituted or unsubstituted C6 to C20 arylene group, a substituted or unsubstituted C1 to C20 heteroarylene group, or one or more combinations thereof,

M¹ to M⁵ may each independently be a single bond, —O—, —S(═O)—, —S(═O)₂—, —C(═O)—, —(CO)O—, —O(CO)O—, —NR″″— (wherein, R″″ is hydrogen, deuterium, or a C1 to C10 alkyl group), or one or more combinations thereof, and

* may be a linking point.

A in Chemical Formula 2 to Chemical Formula 5 may be represented by at least one of structures represented by Chemical Formula A-1 to Chemical Formula A-4.

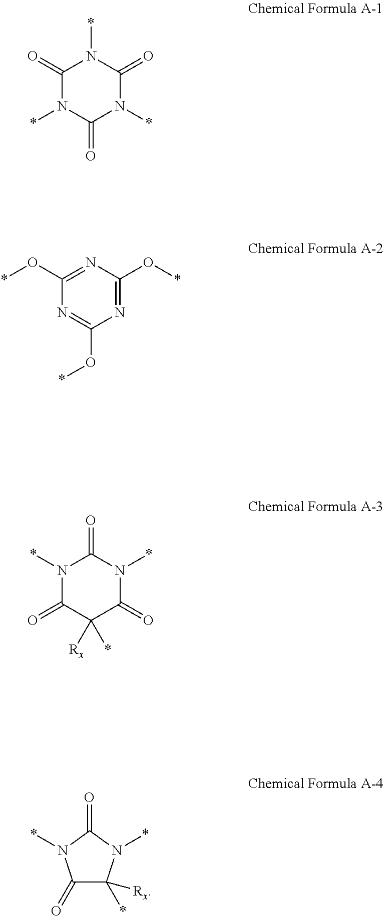

In Chemical Formula A-1 to Chemical Formula A-4,

Rₓ may independently be hydrogen, a substituted or unsubstituted C1 to C10 alkyl group, a substituted or unsubstituted C2 to C10 alkenyl group, a substituted or unsubstituted C2 to C10 alkynyl group, a substituted or unsubstituted C1 to C10 heteroalkyl group, a substituted or unsubstituted C1 to C10 heteroalkenyl group, a substituted or unsubstituted C1 to C10 heteroalkynyl group, a substituted or unsubstituted C3 to C20 cycloalkyl group, a substituted or unsubstituted C2 to C20 heterocycloalkyl group, a substituted or unsubstituted C6 to C20 aryl group, a substituted or unsubstituted C1 to C20 heteroaryl group, or one or more combinations thereof, and

* may be a linking point.

Ar₁ of Chemical Formula 1 may be a group including a substituted or unsubstituted heterocycle selected from Group 1.

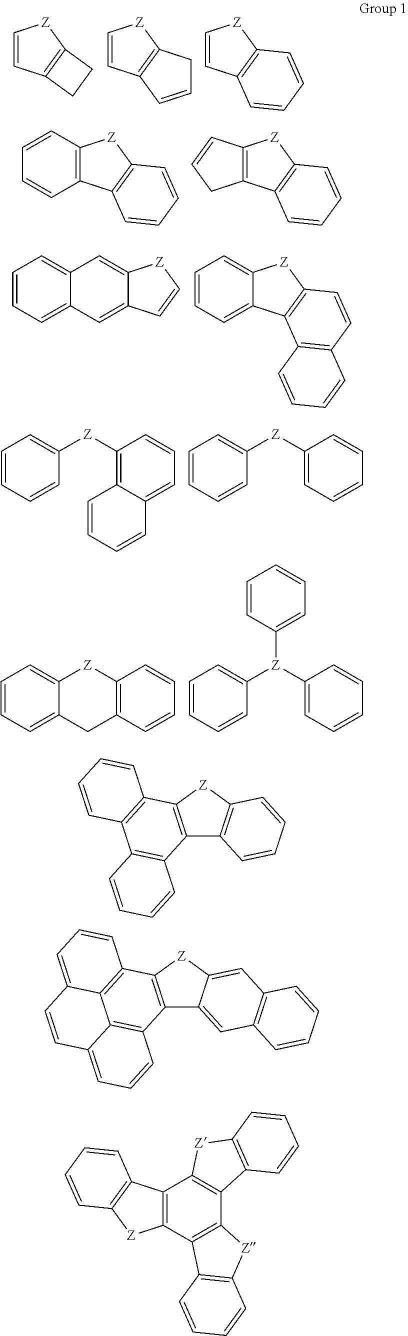

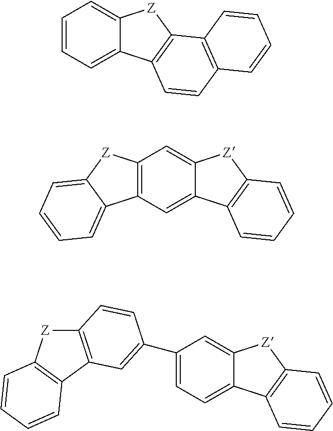

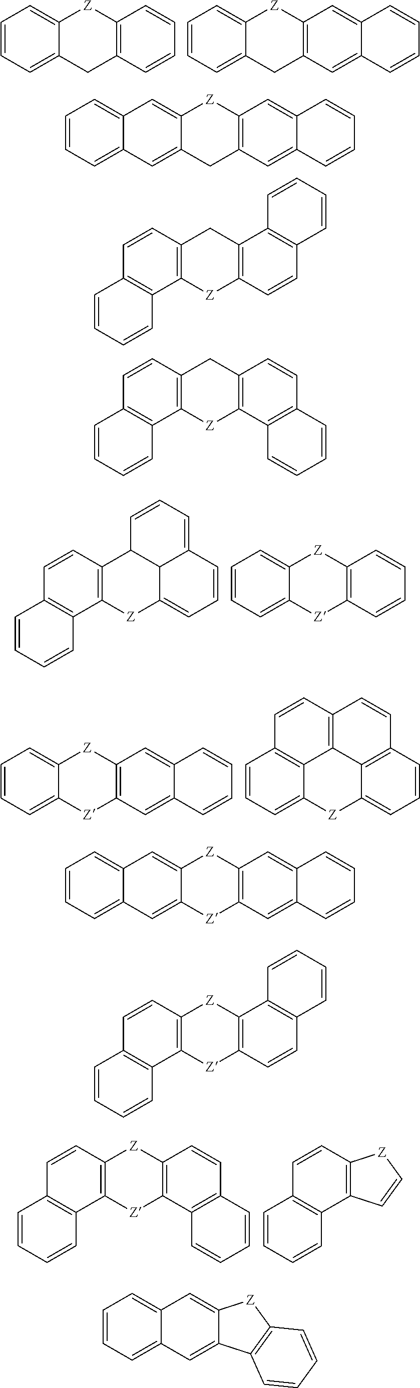

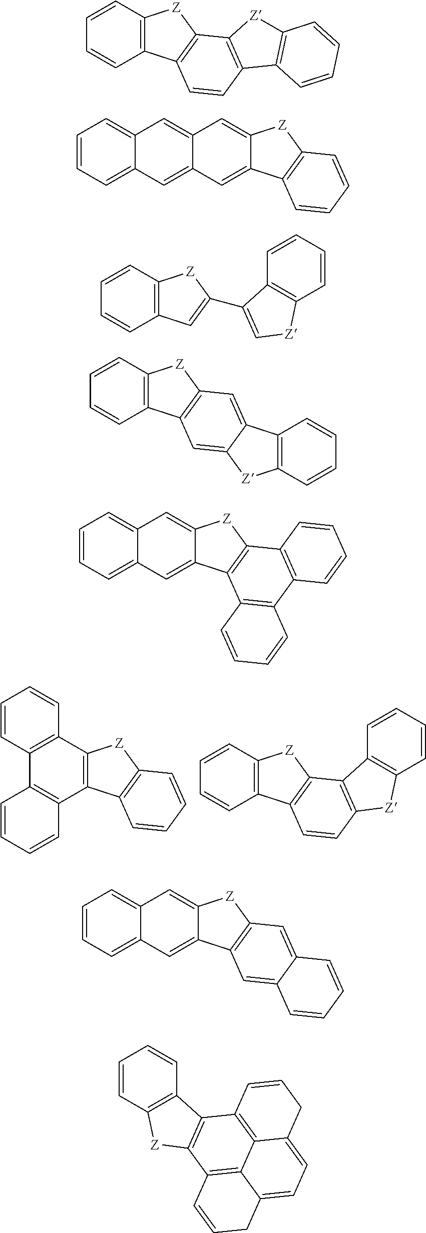

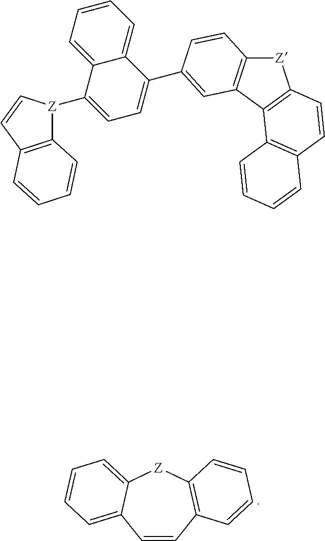

In Group 1,

Z, Z′, and Z″ may each independently be N, O, S, or P, and

the “substituted” may be replaced by a hydroxy group, a C1 to C10 alkoxy group, a cyano group, a halogen atom, a C1 to C30 alkyl group, a C2 to C30 alkenyl group, a C2 to C30 alkynyl group, a C3 to C30 cycloalkenyl group, NR′R″ (wherein R′ and R″ may each independently be hydrogen, a C1 to C10 alkyl group, a C6 to C20 aryl group, or one or more combinations thereof), a C1 to C20 heteroalkyl group, a C2 to C30 heterocycloalkyl group, a C2 to C30 heteroaryl group, or one or more combinations thereof.

Ar₂ of Chemical Formula 1 may be a substituted or unsubstituted aromatic hydrocarbon group selected from Group 2.

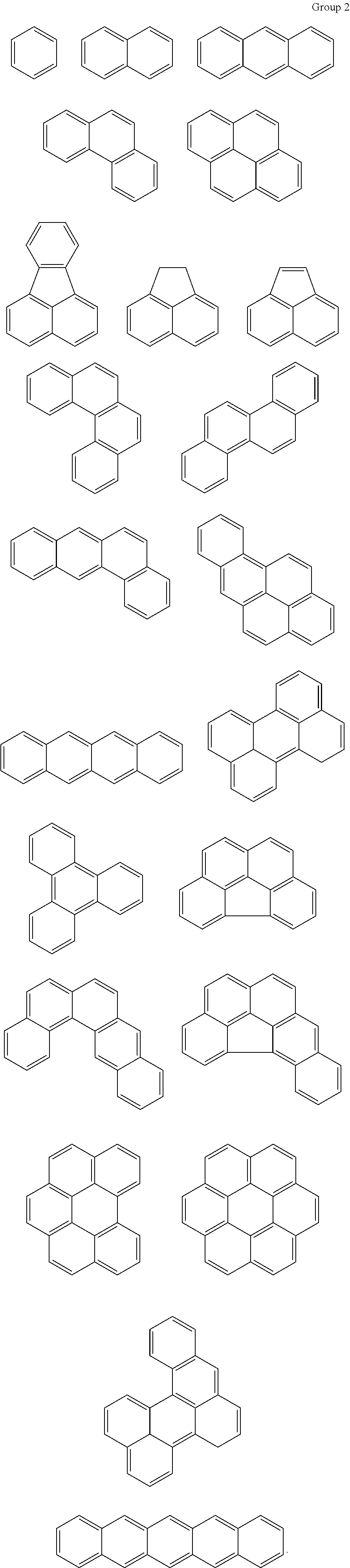

In Group 2,

the “substituted” may be replaced by a hydroxy group, a C1 to C10 alkoxy group, a cyano group, a halogen atom, a C3 to C30 cycloalkenyl group, NR′R″ (wherein, R′ and R″ may each independently be hydrogen, a C1 to C10 alkyl group, a C6 to C20 aryl group, or one or more combinations thereof), a C1 to C20 heteroalkyl group, a C2 to C30 heterocycloalkyl group, a C2 to C30 heteroaryl group, or one or more combinations thereof.

An of Chemical Formula 1 may be a group including a substituted or unsubstituted heterocycle selected from Group 1-1.

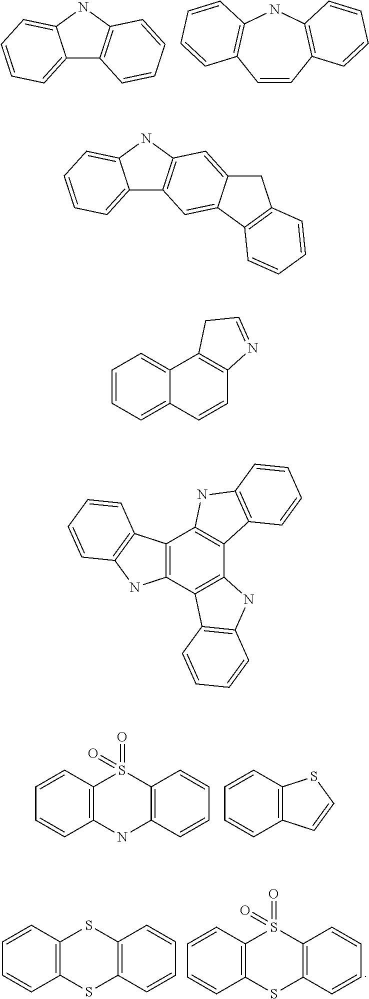

In Group 1-1,

the “substituted” may be replaced by a hydroxy group, a C1 to C10 alkoxy group, a cyano group, a halogen atom, a C1 to C30 alkyl group, a C2 to C30 alkenyl group, a C2 to C30 alkynyl group, a C3 to C30 cycloalkenyl group, NR′R″ (wherein R′ and R″ may each independently be hydrogen, a C1 to C10 alkyl group, a C6 to C20 aryl group, or one or more combinations thereof), a C1 to C20 heteroalkyl group, a C2 to C30 heterocycloalkyl group, a C2 to C30 heteroaryl group, or one or more combinations thereof.

Ar₂ of Chemical Formula 1 may be a substituted or unsubstituted aromatic hydrocarbon group selected from Group 2-1.

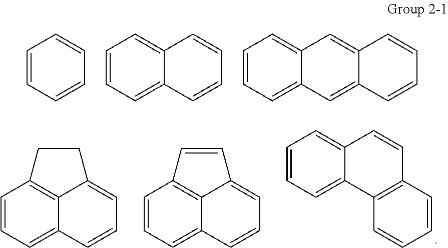

In Group 2-1,

the “substituted” may be replaced by a hydroxy group, a C1 to C10 alkoxy group, a cyano group, a halogen atom, a C3 to C30 cycloalkenyl group, NR′R″ (wherein R′ and R″ may each independently be hydrogen, a C1 to C10 alkyl group, a C6 to C20 aryl group, or one or more combinations thereof), a C1 to C20 heteroalkyl group, a C2 to C30 heterocycloalkyl group, a C2 to C30 heteroaryl group, or one or more combinations thereof.

B₁ and B₂ of Chemical Formula 1 may each independently include a substituted or unsubstituted one selected from Group 3.

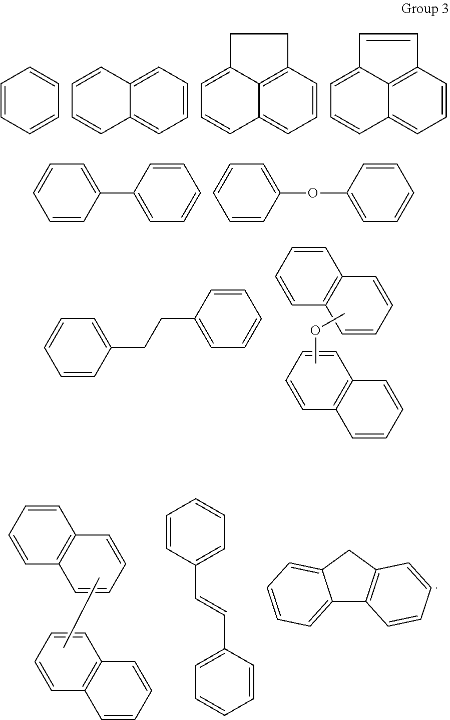

In Group 3,

the “substituted” may be replaced by a hydroxy group, a C1 to C10 alkoxy group, a cyano group, a halogen atom, a C3 to C30 cycloalkenyl group, NR′R″ (wherein R′ and R″ may each independently be hydrogen, a C1 to C10 alkyl group, a C6 to C20 aryl group, or one or more combinations thereof), a C1 to C20 heteroalkyl group, a C2 to C30 heterocycloalkyl group, a C2 to C30 heteroaryl group, or one or more combinations thereof.

A weight ratio of the polymer to the compound included in the resist underlayer composition according to an embodiment may be about 9:1 to about 1:9.

A weight average molecular weight of the polymer may be about 2,000 g/mol to about 300,000 g/mol.

A molecular weight of the compound may be about 300 g/mol to about 5,000 g/mol.

A molecular weight of the compound may be about 1,000 g/mol to about 50,000 g/mol.

The resist underlayer composition may further include one or more polymers selected from among an acrylic resin, an epoxy resin, a novolac resin, a glycoluril-based resin, and a melamine-based resin.

The resist underlayer composition may further include an additive including a crosslinking agent, a thermal acid generator, a surfactant, a plasticizer, or one or more combinations thereof.

According to another embodiment, a method of forming patterns includes forming an etching-objective layer on a substrate, applying (e.g., coating and heat-treating) a resist underlayer composition according to an embodiment on the etching-objective layer to form a resist underlayer, forming a photoresist pattern on the resist underlayer, and sequentially etching the resist underlayer and the etching-objective layer utilizing the photoresist pattern as an etch mask.

The forming of the resist underlayer may further include heat-treating the resist underlayer composition at a temperature of about 100° C. to about 500° C. after coating the resist underlayer composition.

The forming of the photoresist pattern may include forming a photoresist layer on the resist underlayer, exposing the photoresist layer, and developing the photoresist layer.

The resist underlayer composition according to an embodiment does not cause or has reduced pattern collapse of the resist even in a fine patterning process, and is formed into a thin layer to shorten the etching process time, and improves the patterning performance and efficiency by improving sensitivity to the exposure light source.

The resist underlayer composition according to an embodiment may provide a substantially uniform resist underlayer without (or with reduce) phase separation even when different materials are mixed.

Accordingly, the resist underlayer composition according to an embodiment or a resist underlayer prepared therefrom may be advantageously utilized to form a fine pattern of a photoresist utilizing a high energy light source such as EUV (Extreme UltraViolet).

### BRIEF DESCRIPTION OF THE DRAWINGS

FIG. 1 is a cross-sectional view for explaining a method of forming patterns utilizing a resist underlayer composition according to an embodiment.

FIG. 2 is a cross-sectional view for explaining a method of forming patterns utilizing a resist underlayer composition according to an embodiment.

FIG. 3 is a cross-sectional view for explaining a method of forming patterns utilizing a resist underlayer composition according to an embodiment.

FIG. 4 is a cross-sectional view for explaining a method of forming patterns utilizing a resist underlayer composition according to an embodiment.

FIG. 5 is a cross-sectional view for explaining a method of forming patterns utilizing a resist underlayer composition according to an embodiment.

FIG. 6 is a cross-sectional view for explaining a method of forming patterns utilizing a resist underlayer composition according to an embodiment.

### DETAILED DESCRIPTION

The present disclosure may be modified in many alternate forms, and thus specific embodiments will be exemplified in the drawings and described in more detail. It should be understood, however, that it is not intended to limit the present disclosure to the particular forms disclosed, but rather, is intended to cover all modifications, equivalents, and alternatives falling within the spirit and scope of the present disclosure.

Hereinafter, embodiments of the present disclosure will be described in more detail so that those of ordinary skill in the art to which the present disclosure pertains can easily implement them. However, this disclosure may be embodied in many different forms and is not construed as limited to the example embodiments set forth herein.

In the drawings, the thickness of layers, films, panels, regions, etc., may be exaggerated for clarity and like reference numerals designate like elements throughout, and duplicative descriptions thereof may not be provided in the disclosure. It will be understood that when an element such as a layer, film, region, or substrate is referred to as being “on” another element, it can be directly on the other element or intervening element(s) may also be present. In contrast, when an element is referred to as being “directly on” another element, there are no intervening elements present.

As utilized herein, when a definition is not otherwise provided, “substituted” refers to a replacement of a hydrogen atom of a compound by (e.g., the “substituted” may be replaced by) a substituent selected from among a halogen atom (F, Br, Cl, or I), a hydroxy group, an alkoxy group, a nitro group, a cyano group, an amino group, an azido group, an amidino group, a hydrazino group, a hydrazono group, a carbonyl group, a carbamyl group, a thiol group, an ester group, a carboxyl group or a salt thereof, sulfonic acid group or a salt thereof, a phosphoric acid group or a salt thereof, a vinyl group, a C1 to C20 alkyl group, a C2 to C20 alkenyl group, a C2 to C20 alkynyl group, a C6 to C30 aryl group, a C7 to C30 arylalkyl group, a C9 to C30 allylaryl group, a C1 to C30 alkoxy group, a C1 to C20 heteroalkyl group, a C3 to C20 heteroarylalkyl group, a C3 to C30 cycloalkyl group, a C3 to C15 cycloalkenyl group, a C6 to C15 cycloalkynyl group, a C3 to C30 heterocycloalkyl group, and one or more combinations thereof.

In some embodiments, two adjacent substituents of the substituted halogen atom (F, Br, Cl, or I), hydroxy group, nitro group, cyano group, amino group, azido group, amidino group, hydrazino group, hydrazono group, carbonyl group, carbamyl group, thiol group, ester group, carboxyl group or salt thereof, sulfonic acid group or salt thereof, phosphoric acid group or salt thereof, C1 to C30 alkyl group, C2 to C30 alkenyl group, C2 to C30 alkynyl group, C6 to C30 aryl group, C7 to C30 arylalkyl group, C1 to C30 alkoxy group, C1 to C20 heteroalkyl group, C3 to C20 heteroarylalkyl group, C3 to C30 cycloalkyl group, C3 to C15 cycloalkenyl group, C6 to C15 cycloalkynyl group, or C2 to C30 heterocyclic group may be fused to form a ring. For example, the substituted C6 to C30 aryl group may be fused with another adjacent substituted C6 to C30 aryl group to form a substituted or unsubstituted fluorene ring.

In the present disclosure, when a definition is not otherwise provided, “aliphatic hydrocarbon group” includes “saturated aliphatic hydrocarbon group” and “unsaturated aliphatic hydrocarbon group.”

The “saturated aliphatic hydrocarbon group” includes a functional group in which all bonds between carbons are single bonds, for example, an alkyl group or an alkylene group. In some embodiments, the “unsaturated aliphatic hydrocarbon group” refers to a functional group in which an intercarbon bond includes one or more unsaturated bonds, and may include, for example, a double bond or a triple bond, for example, an alkenyl group, an alkynyl group, an alkenylene group, or an alkynylene group.

As utilized herein, when a definition is not otherwise provided, “aromatic hydrocarbon group” refers to a group having one or more hydrocarbon aromatic moieties, in which hydrocarbon aromatic moieties are linked by a single bond and hydrocarbon aromatic moieties are directly or indirectly fused with non-aromatic fused rings.

As utilized herein, “heterocyclic group” includes a heteroaryl group, and may include at least one hetero atom selected from among N, O, S, P, and Si instead of carbon (C) in a cyclic compound such as an aryl group, a cycloalkyl group, a fused ring thereof, or one or more combinations thereof. When the heterocyclic group is a fused ring, the entire ring or each ring of the heterocyclic group may include one or more heteroatoms.

For example, the substituted or unsubstituted aryl group and/or a substituted or unsubstituted heterocyclic group may be a substituted or unsubstituted phenyl group, a substituted or unsubstituted naphthyl group, a substituted or unsubstituted anthracenyl group, a substituted or unsubstituted phenanthryl group, a substituted or unsubstituted naphthacenyl group, a substituted or unsubstituted pyrenyl group, a substituted or unsubstituted biphenyl group, a substituted or unsubstituted terphenyl group, a substituted or unsubstituted quaterphenyl group, a substituted or unsubstituted chrysenyl group, a substituted or unsubstituted triphenylenyl group, a substituted or unsubstituted perylenyl group, a substituted or unsubstituted indenyl group, a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridinyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted fluorenyl group, a substituted or unsubstituted dibenzofuranyl group, a substituted or unsubstituted dibenzothiphenyl group, a substituted or unsubstituted carbazolyl group, pyridoindolyl group, benzopyridooxazinyl group, benzopyridothiazinyl group, 9,9-dimethyl 9,10-dihydroacridinyl group, one or more combinations thereof, or a combined fused ring of the foregoing groups, but are not limited thereto. In one example of the present disclosure, the heterocyclic group or the heteroaryl group may be a pyridyl group, an indolyl group, a carbazolyl group, or a pyridoindolyl group.

As utilized herein, when specific definition is not otherwise provided, the term “combination” refers to mixing or copolymerization.

As utilized herein, the polymer is meant to include an oligomer and a polymer.

Unless otherwise specified in the present disclosure, the weight average molecular weight is measured by dissolving a powder sample in tetrahydrofuran (THF) and then utilizing 1200 series Gel Permeation Chromatography (GPC) of Agilent Technologies (column is Shodex Company LF-804, standard sample is Shodex company polystyrene).

In some embodiments, unless otherwise defined in the disclosure, “*” indicates a linking point of a structural unit or a compound moiety of a compound.

A lithographic technique includes a process of forming a layer with a resist material on a substrate, selectively exposing it to a specific light source by utilizing a mask for forming a set or predetermined pattern thereon, and developing it to form a pattern on a resist layer. In contrast, a demand for reducing a chip size is in a trend (e.g., constant or inconstant trend) in the semiconductor industry. In order to respond to this trend, a resist patterned in the lithographic technique should have a line width reduced to tens of nanometers, so that the formed pattern may be transferred to an underlayer material in the lower substrate through an etching process. However, because the resist has a smaller pattern size, a height (an aspect ratio) of the resist capable of withstanding the line width is limited and thus may not have sufficient resistance during the etching. In this regard, when a resist material needs to be thin, a thick substrate is utilized for etching, or a deep pattern is required, a resist underlayer has been utilized to compensate for these.

This resist underlayer acts as a second mask between the resist layer and the substrate for the patterning and thus should withstand the etching process required during the pattern transferring. Concurrently (e.g., simultaneously), the resist underlayer should be formed with a thin thickness so as to shorten exposure time in the etching process. As the resist underlayer becomes thinner, it becomes even more important to substantially uniformly coat the resist underlayer, wherein when two or more components, having different characteristics from each other, are included in the resist underlayer composition, there may be unfavorable phenomena in forming an ultra-thin film, such as phase-separation, coating non-uniformity, and/or the like.

The Inventors of the present disclosure provide a substantially uniform resist underlayer composition including two or more components with different characteristics to maintain advantageous effects of each component and solve the above problems, that is, phase-separation, coating non-uniformity, and/or the like due to the combination of the different components, so that this composition demonstrates all the excellent or suitable effects of each component, and have confirmed that an ultra-thin layer formed thereof has an excellent or suitable film density and also, a high etch performance and sensitivity.

For example, the resist underlayer composition according to an embodiment includes a polymer including a main chain, a side chain, or a main chain and a side chain including a heterocycle including two or more nitrogen atoms in the ring, a compound including a moiety represented by Chemical Formula 1, and a solvent.

The polymer included in the resist underlayer composition according to an embodiment may include any one or more of the structural units represented by Chemical Formula 2 to Chemical Formula 5.

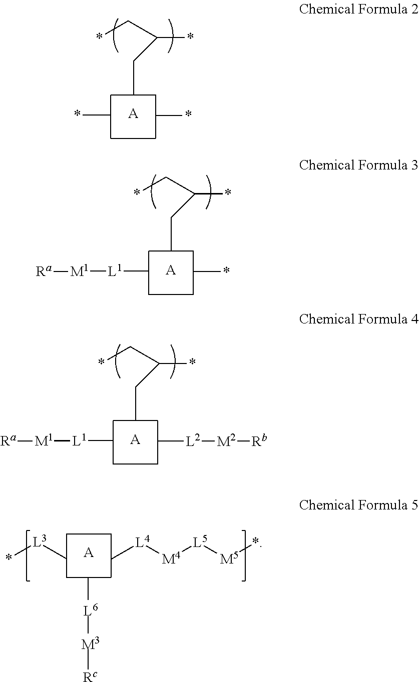

In Chemical Formula 2 to Chemical Formula 5,

A may be a heterocycle containing two or more nitrogen atoms in the ring,

R^{a}, R^{b}, and R^{c} may each independently be a hydroxy group, a substituted or unsubstituted C1 to C10 alkyl group, a substituted or unsubstituted C2 to C10 alkenyl group, a substituted or unsubstituted C2 to C10 alkynyl group, a substituted or unsubstituted C1 to C10 heteroalkyl group, a substituted or unsubstituted C1 to C10 heteroalkenyl group, a substituted or unsubstituted C1 to C10 heteroalkynyl group, a substituted or unsubstituted C3 to C20 cycloalkyl group, a substituted or unsubstituted C2 to C20 heterocycloalkyl group, a substituted or unsubstituted C6 to C20 aryl group, a substituted or unsubstituted C1 to C20 heteroaryl group, or one or more combinations thereof,

L¹ to L⁵ may each independently be a single bond, a substituted or unsubstituted C1 to C10 alkylene group, a substituted or unsubstituted C1 to C10 heteroalkylene group, a substituted or unsubstituted C3 to C20 cycloalkylene group, a substituted or unsubstituted C2 to C20 heterocycloalkylene group, a substituted or unsubstituted C6 to C20 arylene group, a substituted or unsubstituted C1 to C20 heteroarylene group, or one or more combinations thereof,

M¹ to M⁵ may each independently be a single bond, —O—, —S—, —S(═O)—, —S(═O)₂—, —C(═O)—, —(CO)O—, —O(CO)O—, —NR″″— (wherein, R″″ is hydrogen, deuterium, or a C1 to C10 alkyl group), or one or more combinations thereof, and

* is a linking point.

In an embodiment, “A” in Chemical Formula 2 and Chemical Formula 3 may be represented by at least one of the structures represented by Chemical Formula A-1 to Chemical Formula A-4.

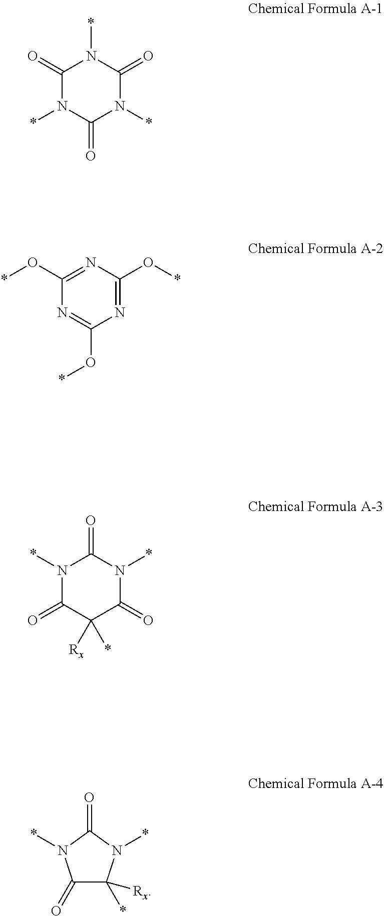

In Chemical Formula A-1 to Chemical Formula A-4,

Rₓ may independently be a hydrogen, a substituted or unsubstituted C1 to C10 alkyl group, a substituted or unsubstituted C2 to C10 alkenyl group, a substituted or unsubstituted C2 to C10 alkynyl group, a substituted or unsubstituted C1 to C10 heteroalkyl group, a substituted or unsubstituted C1 to C10 heteroalkenyl group, a substituted or unsubstituted C1 to C10 heteroalkynyl group, a substituted or unsubstituted C3 to C20 cycloalkyl group, a substituted or unsubstituted C2 to C20 heterocycloalkyl group, a substituted or unsubstituted C6 to C20 aryl group, a substituted or unsubstituted C1 to C20 heteroaryl group, or one or more combinations thereof, and

* is a linking point.

As described above, because A in the structural units represented by Chemical Formula 2 and Chemical Formula 3 of the polymer includes a heterocycle containing two or more nitrogen atoms in the ring, the resist underlayer derived from the resist underlayer composition including the polymer may improve (increase) a density.

In some embodiments, Chemical Formula A-1 to Chemical Formula A-4 include a functional group including a triazine or isocyanurate backbone, and a double bond to the triazine or isocyanurate backbone. The functional group including the double bond has a high electron density because it is possible to be bonded with a triazine or isocyanurate backbone by a sp²-sp² bond, and thus it is possible to improve (increase) the density of the thin layer to implement a layer having a dense structure in the form of an ultra-thin layer, and to improve (increase) light absorption efficiency during exposure of the resist underlayer composition.

In some embodiments, when the resist underlayer is formed utilizing the resist underlayer composition according to an embodiment, secondary electrons may be additionally generated during the photo process, and the additionally generated secondary electrons are transferred to the photoresist during the photo process to maximize or increase an acid generation efficiency. Accordingly, sensitivity of the photoresist may be improved by increasing a photo-processing speed of the photoresist.

In some embodiments, etch selectivity is improved due to the triazine backbone, and energy efficiency may be improved when the pattern is formed after exposure to high energy rays such as EUV (Extreme UltraViolet; wavelength 13.5 nm) and E-beam (electron beam).

In some embodiments, the compound included in the resist underlayer composition according to an embodiment includes a moiety represented by Chemical Formula 1.

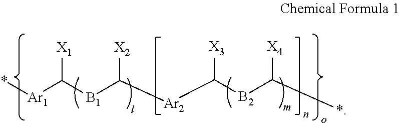

In Chemical Formula 1,

Ar₁ may be a group including a heterocycle,

Ar₂ may be a substituted or unsubstituted C6 to C30 aromatic hydrocarbon group,

B₁ and B₂ may each independently be a single bond, a substituted or unsubstituted C1 to C10 aliphatic hydrocarbon group, a substituted or unsubstituted C6 to C30 aromatic hydrocarbon group, or one or more combinations thereof,

X₁ to X₄ may each independently be hydrogen, deuterium, a hydroxy group, a thiol group, a cyano group, a substituted or unsubstituted amino group, a halogen atom, a substituted or unsubstituted C1 to C30 aliphatic hydrocarbon group, a substituted or unsubstituted C1 to C30 alkoxy group, a substituted or unsubstituted C6 to C30 aromatic hydrocarbon group, a substituted or unsubstituted C2 to C30 heterocyclic group, or one or more combinations thereof,

I, m, and n may each independently be an integer from 0 to 5, and

o may be an integer from 1 to 30.

In an embodiment, Ar₁ of Chemical Formula 1 may include a substituted or unsubstituted heterocycle selected from Group 1.

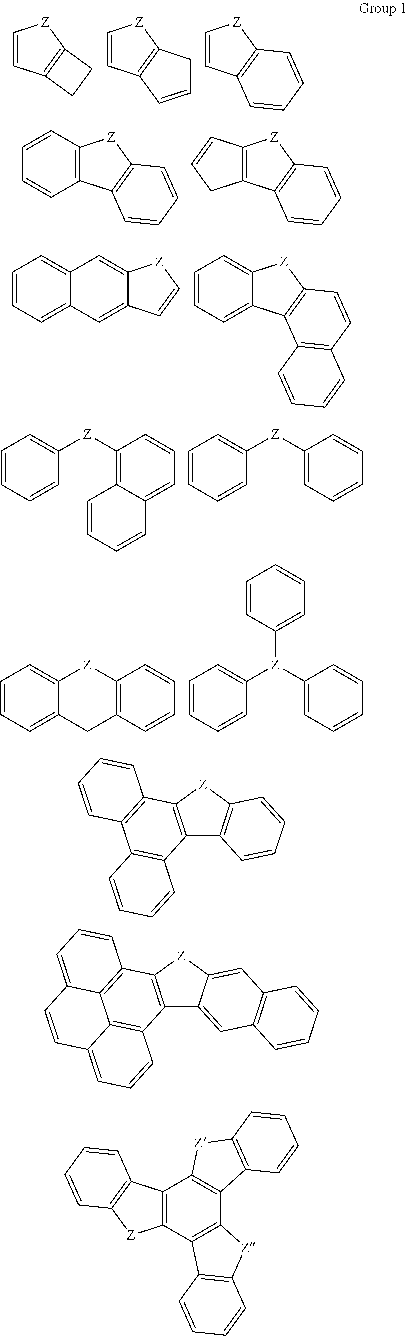

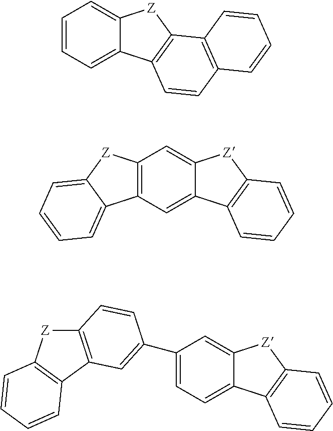

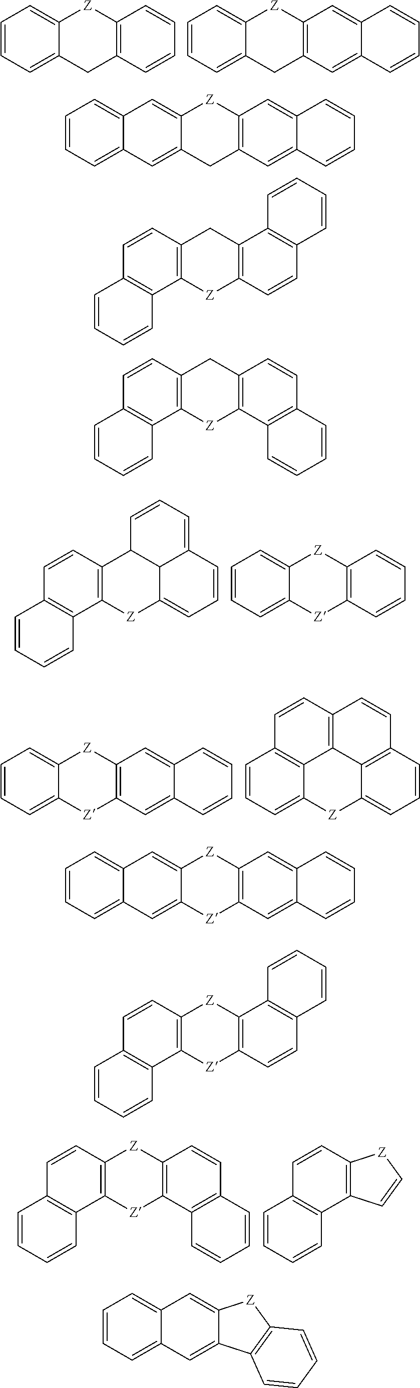

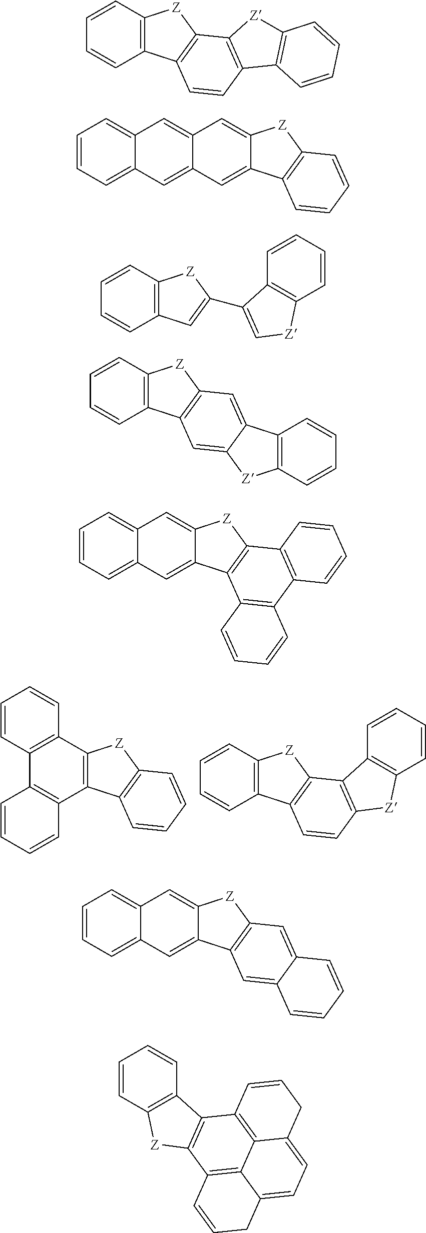

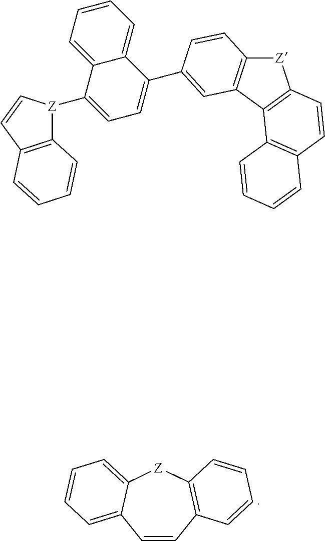

In Group 1,

Z, Z′, and Z″ may each independently be N, O, S, or P, and

The heterocycle selected from Group 1 may be substituted with a hydroxy group, a C1 to C10 alkoxy group, a cyano group, a halogen atom, a C1 to C30 alkyl group, a C2 to C30 alkenyl group, a C2 to C30 alkynyl group, a C3 to C30 cycloalkenyl group, NR′R″ (wherein R′ and R″ may each independently be hydrogen, a C1 to C10 alkyl group, a C6 to C20 aryl group, or one or more combinations thereof.), a C1 to C20 heteroalkyl group, a C2 to C30 heterocycloalkyl group, a C2 to C30 heteroaryl group, or one or more combinations thereof.

In another embodiment, Ar₁ in Chemical Formula 1 may include a substituted or unsubstituted hetero ring selected from Group 1-1.

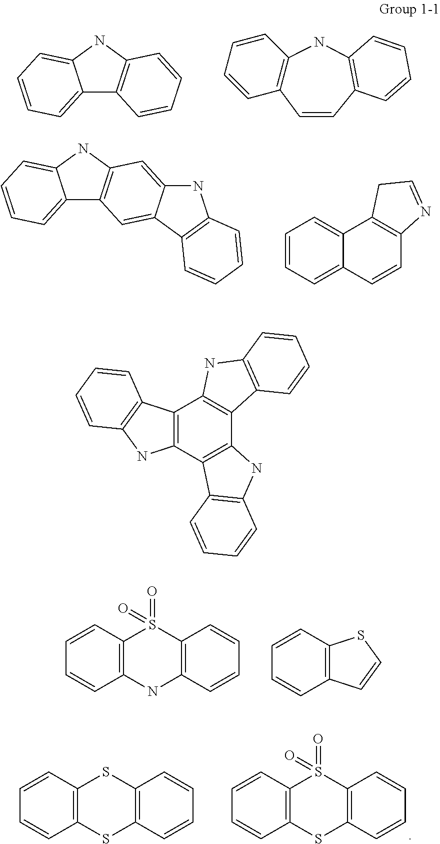

The group including a heterocycle selected from Group 1-1 may be substituted with a hydroxy group, a C1 to C10 alkoxy group, a cyano group, a halogen atom, a C1 to C30 alkyl group, a C2 to C30 alkenyl group, a C2 to C30 alkynyl group, a C3 to C30 cycloalkenyl group, NR′R″ (wherein R′ and R″ may each independently be hydrogen, a C1 to C10 alkyl group, a C6 to C20 aryl group, or one or more combinations thereof.), a C1 to C20 heteroalkyl group, a C2 to C30 heterocycloalkyl group, a C2 to C30 heteroaryl group, or one or more combinations thereof. For example, it may be substituted with a hydroxy group, a C1 to C30 alkyl group, or a C2 to C30 alkenyl group.

In one embodiment, Ar₂ of Chemical Formula 1 may be a substituted or unsubstituted aromatic hydrocarbon group selected from Group 2.

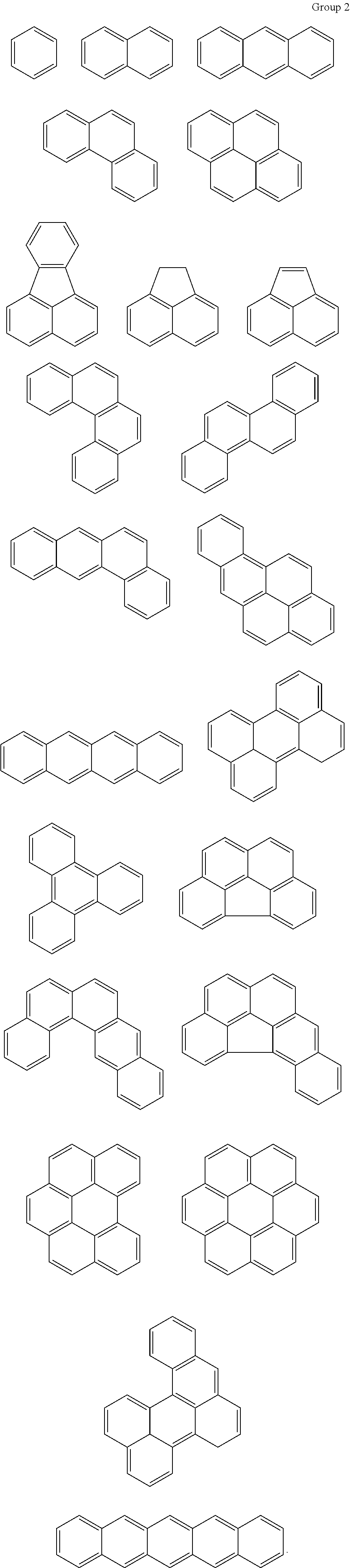

The aromatic hydrocarbon group selected from Group 2 may be substituted with a hydroxy group, a C1 to C10 alkoxy group, a cyano group, a halogen atom, a C3 to C30 cycloalkenyl group, NR′R″ (wherein, R′ and R″ may each independently be hydrogen, a C1 to C10 alkyl group, a C6 to C20 aryl group, or one or more combinations thereof), a C1 to C20 heteroalkyl group, a C2 to C30 heterocycloalkyl group, a C2 to C30 heteroaryl group, or one or more combinations thereof. For example, it may be substituted with a hydroxy group or a C1 to C10 alkoxy group.

In an embodiment, Ar₂ of Chemical Formula 1 may be a substituted or unsubstituted aromatic hydrocarbon group selected from Group 2-1 among the substituted or unsubstituted aromatic hydrocarbon groups selected from Group 2.

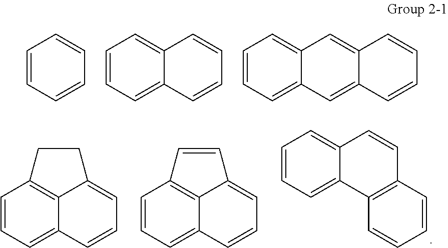

The aromatic hydrocarbon group selected from Group 2-1, as in Group 2, may be substituted with a hydroxy group, a C1 to C10 alkoxy group, a cyano group, a halogen atom, a C3 to C30 cycloalkenyl group, NR′R″ (wherein, R′ and R″ may each independently be hydrogen, a C1 to C10 alkyl group, a C6 to C20 aryl group, or one or more combinations thereof), a C1 to C20 heteroalkyl group, a C2 to C30 heterocycloalkyl group, a C2 to C30 heteroaryl group, or one or more combinations thereof. For example, it may be substituted with a hydroxy group or a C1 to C10 alkoxy group.

In one embodiment, B₁ and B₂ in Chemical Formula 1 may each independently include a substituted or unsubstituted cyclic compound selected from Group 3.

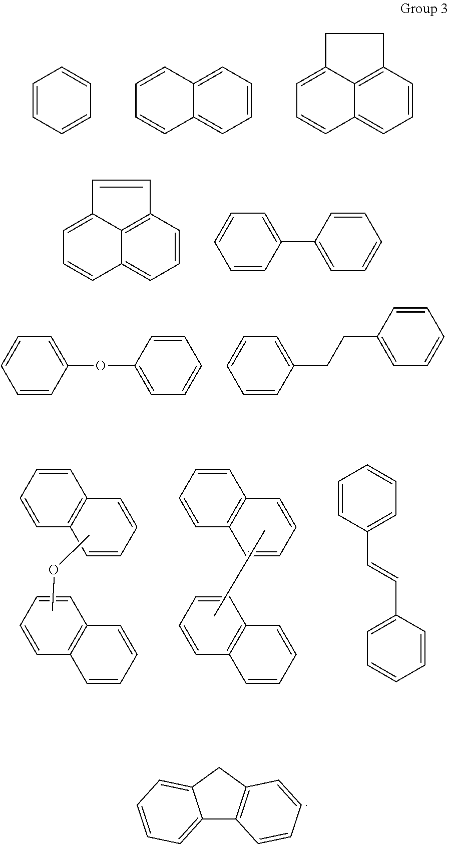

The cyclic compound selected from Group 3 may be substituted with a hydroxy group, a C1 to C10 alkoxy group, a cyano group, a halogen atom, a C3 to C30 cycloalkenyl group, NR′R″ (wherein R′ and R″ may each independently be hydrogen, a C1 to C10 alkyl group, a C6 to C20 aryl group, or one or more combinations thereof.), a C1 to C20 heteroalkyl group, a C2 to C30 heterocycloalkyl group, a C2 to C30 heteroaryl group, or one or more combinations thereof.

In an embodiment, B₁ and B₂ of Chemical Formula 1 may be a substituted or unsubstituted cyclic compound selected from Group 3-1 among the substituted or unsubstituted aromatic hydrocarbon groups selected from Group 3.

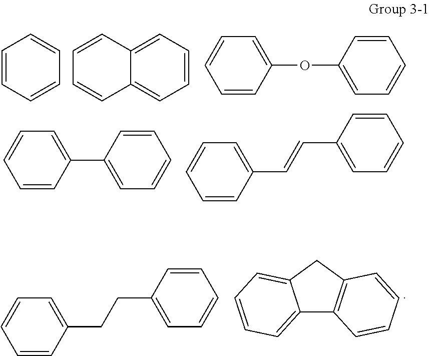

The cyclic compound selected from Group 3-1, as in Group 3, may be substituted with a hydroxy group, a C1 to C10 alkoxy group, a cyano group, a halogen atom, a C3 to C30 cycloalkenyl group, NR′R″ (wherein R′ and R″ may each independently be hydrogen, a C1 to C10 alkyl group, a C6 to C20 aryl group, or one or more combinations thereof.), a C1 to C20 heteroalkyl group, a C2 to C30 heterocycloalkyl group, a C2 to C30 heteroaryl group, or one or more combinations thereof.

A weight ratio of the polymer to the compound included in the resist underlayer composition according to an embodiment may be about 9:1 to about 1:9. For example, the weight ratio may be from about 8:2 to about 2:8, for example from about 7:3 to about 3:7, for example from about 6:4 to about 4:6, but is not limited thereto. In an embodiment, the weight ratio of the polymer to the compound may be about 9:1 to about 1:1, for example, about 8:1 to about 1:1, for example, about 7:1 to about 1:1, for example, about 6:1 to about 1:1, for example, about 5:1 to about 1:1, for example, about 4:1 to about 1:1, or for example, about 3:1 to about 1:1, but is not limited thereto.

By including the polymer and the compound in the composition within the above ranges, thickness, surface roughness, and planarization degree of the resist underlayer may be controlled or selected.

The polymer may have a weight average molecular weight of about 1,000 g/mol to about 300,000 g/mol. For example, the polymer may have a weight average molecular weight of about 2,000 g/mol to about 300,000 g/mol, for example about 2,000 g/mol to about 200,000 g/mol, for example about 2,000 g/mol to about 100,000 g/mol, for example about 2,000 g/mol to about 90,000 g/mol, for example about 2,000 g/mol to about 70,000 g/mol, for example about 2,000 g/mol to about 50,000 g/mol, for example about 2,000 g/mol to about 30,000 g/mol, for example about 2,000 g/mol to about 20,000 g/mol, or for example about 2,000 g/mol to about 10,000 g/mol, but is not limited thereto. By having a weight average molecular weight in the above range, the carbon content (e.g., amount) and solubility in a solvent of the resist underlayer composition including the polymer may be adjusted and may be optimized.

The compound may be a monomolecular compound or an oligomer, and the molecular weight of the compound may be about 300 g/mol to about 5,000 g/mol.

The compound may be a polymer, and the molecular weight (weight average molecular weight) of the compound may be about 1,000 g/mol to about 50,000 g/mol.

When the compound is a monomolecule or oligomer, the molecular weight of the compound may be for example, about 300 g/mol to about 5,000 g/mol, for example about 300 g/mol to about 3,000 g/mol, for example about 500 g/mol to about 3,000 g/mol, for example, about 500 g/mol to about 2,000 g/mol, for example, about 500 g/mol to about 1,500 g/mol, and when the compound is a polymer, the weight average molecular weight of the compound may be for example, about 1,000 g/mol to about 50,000 g/mol, for example about 1,000 g/mol to about 30,000 g/mol, for example about 1,000 g/mol to about 20,000 g/mol, or for example about 1,000 g/mol to about 10,000 g/mol, but is not limited thereto. When the compound including the moiety represented by Chemical Formula 1 has a molecular weight or a weight average molecular weight within the above ranges, a film density of the prepared resist underlayer may be improved, and thus damage or collapse of the photoresist pattern may be prevented or reduced during the patterning process.

The resist underlayer composition according to an embodiment may include a solvent. The solvent is not limited as long as it has sufficient (suitable) solubility and/or dispersibility for the polymer and compound according to an embodiment. The solvent may include, for example propylene glycol, propylene glycol diacetate, methoxypropanediol, diethylene glycol, diethylene glycol butylether, tri(ethylene glycol)monomethylether, propylene glycol monomethylether, propylene glycol monomethylether acetate, cyclohexanone, ethyllactate, gamma-butyrolactone, N,N-dimethyl formamide, N,N-dimethyl acetamide, methylpyrrolidone, methylpyrrolidinone, methyl 2-hydroxyisobutyrate, acetylacetone, ethyl 3-ethoxypropionate, or one or more combinations thereof, but is not limited thereto.

The resist underlayer composition according to an embodiment may further include one or more polymers selected from among an acrylic resin, an epoxy resin, a novolac resin, a glycoluril-based resin, and a melamine-based resin in addition to the polymer, compound, and solvent, but is not limited thereto.

The resist underlayer composition according to another embodiment may further include an additive including a crosslinking agent, a thermal acid generator, a surfactant, a plasticizer, or one or more combinations thereof, but is not limited thereto.

The crosslinking agent may be utilized to further harden the underlayer by inducing a crosslinking reaction, and may include for example, a melamine-based, substituted urea-based, and/or a polymer-based crosslinking agent. In some embodiments, it may be a crosslinking agent having at least two crosslinking substituents and may be, for example, a compound such as methoxymethylated glycoluril, butoxymethylated glycoluril, methoxymethylated melamine, butoxymethylated melamine, methoxymethylated benzoguanamine, butoxymethylated benzoguanamine, methoxymethylated urea, butoxymethylated urea, methoxymethylated thiourea, and/or butoxymethylated thiourea, and/or the like, but the present disclosure is not limited thereto.

As the crosslinking agent, a crosslinking agent having high heat resistance may be utilized, and for example, a compound containing a crosslinking substituent having an aromatic ring (e.g., a benzene ring or a naphthalene ring) in a molecule may be utilized. The crosslinking agent may have, for example, two or more crosslinking sites.

The thermal acid generator may be, for example, an acidic compound such as p-toluenesulfonic acid, trifluoromethanesulfonic acid, pyridinium p-toluenesulfonate, salicylic acid, sulfosalicylic acid, citric acid, benzoic acid, hydroxybenzoic acid, naphthalenecarbonic acid, and/or the like and/or benzointosylate, 2-nitrobenzyltosylate, and/or other organic sulfonic acid alkyl esters, but is not limited thereto.

The surfactant may be utilized to improve coating defects caused by an increase in the solid content (e.g., amount) when forming the resist underlayer, and may be, for example, an alkylbenzenesulfonate salt, an alkylpyridinium salt, a polyethylene glycol, and/or a quaternary ammonium salt, but the present disclosure is not limited thereto.

The plasticizer is not limited, and one or more suitable generally used/generally available plasticizers may be utilized. Examples of the plasticizer may include low molecular weight compounds such as phthalic acid esters, adipic acid esters, phosphoric acid esters, trimellitic acid esters, citric acid esters, and/or the like, polyether compounds, polyester-based compounds, polyacetal compounds, and/or the like.

The additive may be included in an amount of about 0.001 parts to about 40 parts by weight based on 100 parts by weight of the resist underlayer composition. Within the above range, solubility may be improved without changing the optical properties of the resist underlayer composition.

According to another embodiment, a resist underlayer manufactured utilizing the aforementioned resist underlayer composition is provided. The resist underlayer may be in a form that is cured through a heat treatment process after coating the aforementioned resist underlayer composition on, for example, a substrate.

Hereinafter, a method of forming patterns utilizing the aforementioned resist underlayer composition will be described in more details with reference to FIGS. 1 to 6.

FIGS. 1 to 6 are cross-sectional views explaining/illustrating a method of forming patterns utilizing a resist underlayer composition according to an embodiment.

Referring to FIG. 1, first, an object to be etched is prepared. An example of the object to be etched may be the thin layer 102 formed on the semiconductor substrate 100. Hereinafter, the embodiment in which the object to be etched is the thin layer 102 will be described in more detail. A surface of the thin layer 102 is washed to remove impurities and/or the like remaining thereon. The thin layer 102 may be, for example, a silicon nitride, polysilicon layer or silicon oxide layer.

Subsequently, on the surface of the washed thin layer 102, the aforementioned resist underlayer composition may be applied by a spin coating method.

Then, the coated composition is dried and baked (e.g., heat-treated) to form a resist underlayer 104 on the thin layer. The baking (e.g., heat-treating or heating) may be performed at about 100° C. to about 500° C., for example, about 100° C. to about 300° C. For example, the resist underlayer composition is described above in more detail and thus will not be provided again.

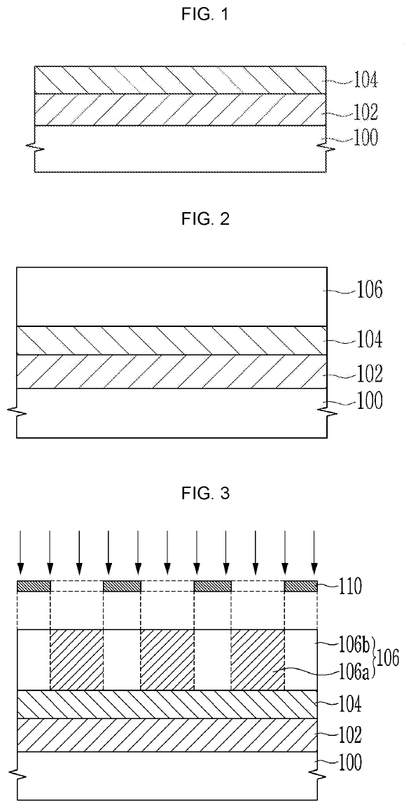

Referring to FIG. 2, a photoresist layer 106 is formed by coating a photoresist on the resist underlayer 104.

Examples of the photoresist may be a positive-type or kind photoresist containing a naphthoquinone diazide compound and a novolac resin; a chemically amplified positive-type or kind photoresist including an acid generator capable of dissociating an acid upon exposure, a compound with increased solubility in alkaline aqueous solution by decomposition in the presence of acid, and an alkali-soluble resin; or a chemically amplified positive-type or kind photoresist including an acid generator and an alkali-soluble resin having a group capable of imparting a resin having increased solubility in aqueous alkali solution by decomposition in the presence of acid.

Then, a substrate 100 having the photoresist layer 106 is initially baked (e.g., heat-treated). The initial baking (e.g., heating) may be performed at about 90° C. to about 120° C.

Referring to FIG. 3, the photoresist layer 106 may be selectively exposed.

Exposure of the photoresist layer 106 may be, for example, performed by positioning an exposure mask having a set or predetermined pattern on a mask stage of an exposure apparatus and aligning the exposure mask 110 on the photoresist layer 106. Then, a set or predetermined region of the photoresist layer 106 formed on the substrate 100 selectively reacts with light passing the exposure mask by radiating light into the exposure mask 110.

Examples of the light utilized during the exposure may include an i-line activating radiation having a wavelength of 365 nm, a KrF excimer laser having a wavelength of 248 nm, and/or a short-wavelength light such as an ArF excimer laser having a wavelength of 193 nm, and/or in addition, extreme ultraviolet (EUV) light having a wavelength of 13.5 nm corresponding to extreme ultraviolet light may be utilized.

The photoresist layer 106a of the exposed portion has greater hydrophilicity compared to the photoresist layer 106b of the unexposed portion. Accordingly, the exposed region 106a and non-exposed region 106b of the photoresist layer 106 may have different solubilities.

Then, the substrate 100 is baked (e.g., heat-treated) for a second time. The second baking may be performed at about 90° C. to about 150° C. The exposed region of the photoresist layer becomes easily soluble (more soluble) with respect to a particular solvent due to the secondary baking.

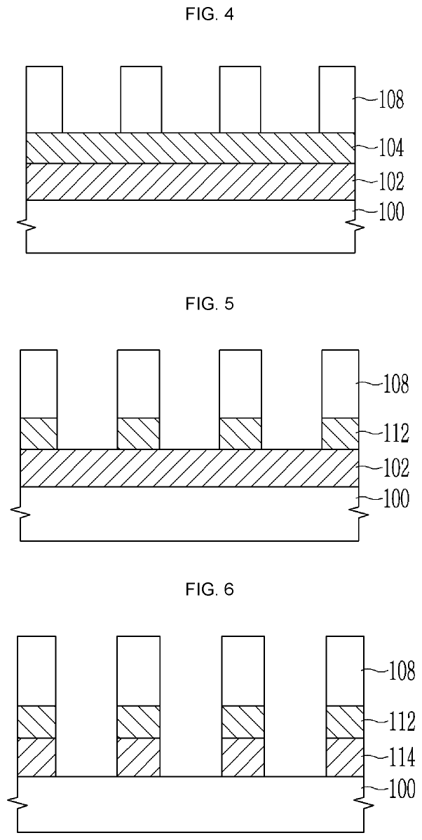

Referring to FIG. 4, for example, by dissolving and removing the photoresist layer corresponding to the exposed region 106a utilizing tetra-methyl ammonium hydroxide (TMAH), etc., the photoresist layer 106b left after development forms a photoresist pattern 108.

Then, the resist underlayer 104 is etched utilizing the photoresist pattern 108 as an etch mask. An organic layer pattern 112 as shown in FIG. 5 is formed by the etching process as described above. The etching may be for example dry etching utilizing etching gas, and the etching gas may be for example CHF₃, CF₄, Cl₂, O₂, and/or a mixed gas thereof. As described above, because the resist underlayer formed by the resist underlayer composition according to the embodiment has a fast etch-rate, a smooth etching process may be performed within a short time.

Referring to FIG. 6, the exposed thin layer 102 is etched by applying the photoresist pattern 108 as an etch mask. As a result, the thin layer is formed into a thin layer pattern 114. In the exposure process performed above, the thin layer pattern 114 formed by the exposure process, performed utilizing a short-wavelength light source such as the activating radiation i-line (wavelength 365 nm), a KrF excimer laser (wavelength 248 nm), and/or an ArF excimer laser (wavelength 193 nm), may have a width of several tens of nm to several hundred nm, and the thin layer pattern 114 formed by the exposure process performed utilizing the EUV light source may have a width of less than or equal to about 20 nm.

Hereinafter, the present disclosure is described in more detail through examples regarding synthesis of the polymer and preparation of a resist underlayer composition including the same. However, the present disclosure is not restricted by the following examples.

### SYNTHESIS EXAMPLES

### Synthesis Example A1

20 g of 1,3-diallyl-5-(2-hydroxyethyl) isocyanurate, 6.7 g of 1,2-ethanedithiol, 1.3 g of AIBN (azobisisobutyronitrile), and 40 g of N,N-dimethyl formamide (DMF) were put in a 500 mL 2-necked round-bottom flask, and a condenser was connected thereto. After proceeding with a reaction at 60° C. for 16 hours, the reaction solution was cooled to room temperature (about 23° C.). The reaction solution was added dropwise to a 1 L wide-mouth bottle containing 800 g of water, while stirred, to form a gum, and the gum was dissolved in 80 g of tetrahydrofuran (THF). The dissolved resin solution was treated with toluene to form precipitates and remove monomolecular compounds and small molecules. Finally, 15 g of a polymer represented by Chemical Formula 3a (a weight average molecular weight (Mw)=7,500 g/mol) was obtained.

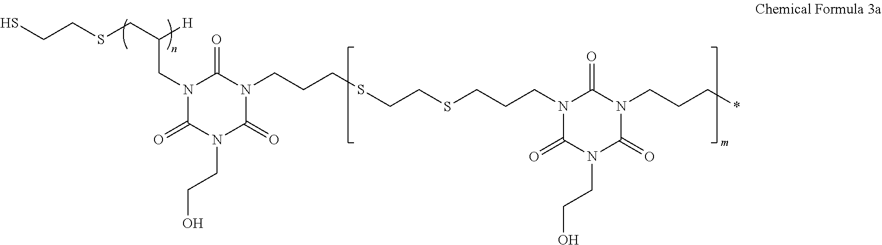

### Synthesis Example A2

24.9 g of 1,3,5-triallyl-1,3,5-triazinane-2,4,6-trione, 7.4 g of 3-mercapto propanol, 0.7 g of AIBN (azobisisobutyronitrile), and 48 g of N,N-dimethyl formamide (DMF) were put in a 500 mL 3-necked round-bottom flask, and a condenser was connected thereto. After proceeding with a reaction at 80° C. for 16 hours, the reaction solution was cooled down to room temperature. The reaction solution was added dropwise in a 1 L wide-mouthed bottle containing 800 g of water, while stirred, to produce gum, and the gum was dissolved in 80 g of tetrahydrofuran (THF). The dissolved resin solution was treated with toluene to form precipitates and remove monomolecular compounds and small molecules. Finally, 10 g of a polymer represented by Chemical Formula 4a (a weight average molecular weight (Mw)=10,500 g/mol).

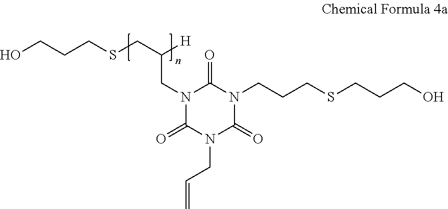

### Synthesis Example A3

148.6 g (0.5 mol) of 1,3,5-triglycidyl isocyanurate, 60.0 g (0.4 mol) of 2,2′-thiodiacetic acid, 9.1 g of benzyl triethyl ammonium chloride, and 350 g of N,N-dimethylformamide were put in a 1 L 2-necked round flask, and a condenser was connected thereto. After increasing the temperature to 100° C., the mixture was reacted for 8 hours and cooled to room temperature (about 23° C.). Subsequently, the reaction solution was transferred to a 1 L wide-mouthed bottle and then, three times washed with hexane and subsequently, with purified water. The obtained resin in a gum state was completely dissolved in 80 g of THF and then, slowly added dropwise to 700 g of toluene. Then, the solvent was removed therefrom, obtaining a polymer including a structural unit represented by Chemical Formula 5a (Mw=9,100 g/mol).

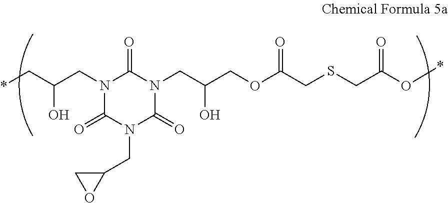

### Synthesis Example B1

24 g of 1,4-benzenedicarbonyl chloride, 50 g of 9-vinyl-9H-carbazole, and 200 g of 1,2-dichloroethane were put in a flask. 34 g of aluminum chloride was slowly added to this solution and then, stirred at room temperature for 6 hours. When a reaction was completed, precipitates formed by adding methanol thereto were filtered and dried, obtaining the intermediate compound. Subsequently, 50 g of the intermediate compound and 200 g of tetrahydrofuran were added to the flask, and 25 g of a sodium borohydride aqueous solution was slowly added thereto and then, stirred at room temperature for 24 hours. When a reaction was completed, a compound therefrom was neutralized to have pH about 7 by utilizing a 2% hydrochloric acid aqueous solution and then, extracted and dried with ethylacetate, obtaining a compound represented by Chemical Formula 1a.

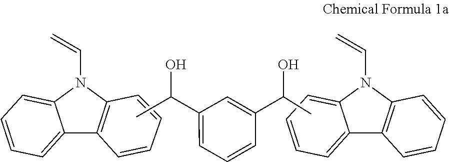

### Synthesis Example B2

In a 500 mL 2-necked flask equipped with a mechanical agitator and a cooling tube, 20 g of 1,1,2-trimethyl-1H-benzoindole, 10.6 g of 10-hydroxyphenanthrene-9-carbaldehyde, 8.6 g of 9-fluorenone, and 4.9 g of p-toluenesulfonic acid were added to 51 g of 1,4-dioxane and then, well stirred, heated to 110° C., and stirred again for 14 hours. When a reaction was completed, after decreasing an internal temperature of the flask to 65° C. and adding 200 g of THF thereto, the resultant was adjusted to have pH about 5 by utilizing a 7% sodium bicarbonate aqueous solution. Subsequently, 1000 mL of ethylacetate was poured thereinto and then, kept being stirred and filtered with a separatory funnel to extract an organic layer alone. Then, 500 mL of water was added to the separatory funnel and then, shaken to remove the remaining acid and sodium salt, which was three times and more repeated, finally extracting the organic layer. Subsequently, the organic solution was concentrated with an evaporator, and 700 g of THF was added thereto the obtained compound, obtaining the compound in a solution state. The solution was slowly added dropwise to a beaker containing 3000 mL of hexane to form precipitates, and the solvent was removed therefrom, obtaining a compound (Mw=1,540 g/mol) including each structural unit represented by Chemical Formulas 1b and 1c.

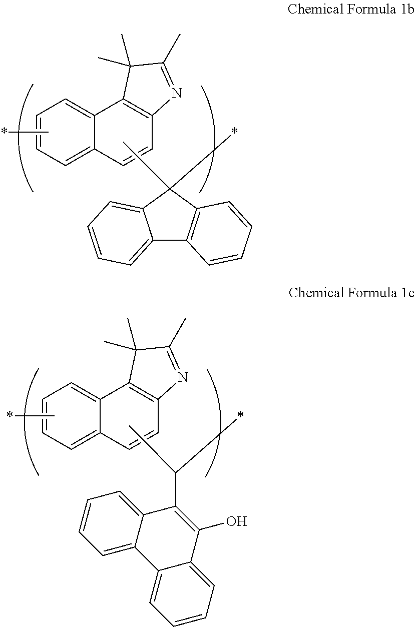

### Synthesis Example B3

10 g of thianaphthene, 10.7 g of 2-naphthol, 40 g of (E)-1,2-bis(4-(methoxymethyl)phenyl)ethene, 18 g of diethyl sulfate, and 15 g of propylene glycol monomethylether acetate (PGMEA) were put in a flask and then, stirred at 110° C. for 8 hours. When a reaction was completed, the resultant was added to 200 g of hexane for precipitation, and precipitates were filtered by adding methanol and water thereto and then, treated with methanol to remove remaining monomers, obtaining a compound (Mw=3,520 g/mol) including each structural unit represented by Chemical Formulas 1d and 1e.

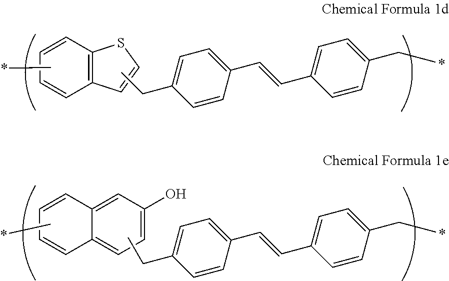

### Synthesis Example B4

20 g of thianthrene, 13.3 g of 2-naphthol, 5.6 g of p-formaldehyde, 1.9 g of p-toluenesulfonic acid, and 50 g of PGMEA were put in a flask and then, stirred at 70° C. for 8 hours. When a reaction was completed, the resultant was added to 200 g of hexane for precipitation, and precipitates were filtered by adding methanol and water thereto and treated with methanol to remove remaining monomers, obtaining a compound (Mw=4,050 g/mol) including a structural unit represented by Chemical Formula 1f.

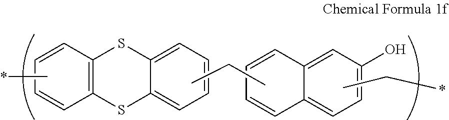

### Preparation of Resist Underlayer Composition

### Examples 1 to 7 and Comparative Examples 1 to 2

0.5 g of the polymer and the compound of each of Examples 1 to 7 (or the polymer of Comparative Example 1 or the compound of Comparative Example 2) according to the Synthesis Examples were (or was) added in a ratio (e.g., amount) shown in Table 1, with 0.125 g of PD1174 (hardener; TCI (Tokyo Chemical Industry)), and 0.01 g of pyridinium para-toluenesulfonate (PPTS), and they were completely or substantially completely dissolved in a mixed solvent of propylene glycol monomethylether and ethyllactate (a mixing volume ratio=7:3), thereby preparing resist underlayer compositions according to Examples 1 to 7 and Comparative Examples 1 to 2, respectively.

TABLE 1

| | Polymer | Compound | Ratio |
|---|---|---|---|
| Example 1 | Synthesis Example A1 | Synthesis Example B1 | 6:4 |
| Example 2 | Synthesis Example A1 | Synthesis Example B2 | 7:3 |
| Example 3 | Synthesis Example A1 | Synthesis Example B4 | 8:2 |
| Example 4 | Synthesis Example A2 | Synthesis Example B1 | 7:3 |
| Example 5 | Synthesis Example A2 | Synthesis Example B2 | 6:4 |
| Example 6 | Synthesis Example A2 | Synthesis Example B3 | 7:3 |
| Example 7 | Synthesis Example A3 | Synthesis Example B4 | 7:3 |
| Comparative | Synthesis Example A1 | — | — |
| Example 1 | | | |
| Comparative | — | Synthesis Example B2 | — |
| Example 2 | | | |

### Evaluation of Etch Resistance

The compositions according to Examples 1 and 5 and Comparative Examples 1 and 2 were respectively taken by 2 mL (2 mL of each composition was removed), cast on an 8-inch wafer, spin-coated at a speed of 1,500 rpm for 20 seconds with an auto track (ACT-8, TEL (Tokyo Electron Limited)), and cured at 210° C. for 90 seconds, forming 50 Å-thick thin layers.

Subsequently, the formed thin layers were dry-etched under CF₄, CHF₃. And O₂ gas for 20 seconds and then, measured with respect to an etched thickness, showing an etch-rate relative to Comparative Example 1. Table 2 shows results obtained by calculating relative etch-rates of the examples or the comparative examples when the etch-rate of Comparative Example 1 was 1.

TABLE 2

| | Etch-rate (Å/sec) |
|---|---|
| Example 1 | 0.8 |
| Example 5 | 0.8 |
| Comparative Example 1 | 1 |
| Comparative Example 2 | 0.6 |

Referring to Table 2, the underlayers respectively formed of the resist underlayer compositions according to Examples 1 and 5 were shown to have sufficient (suitable) etch resistance against the etching gas.

### Evaluation of Coating Uniformity

The compositions according to Examples 1 to 7 and Comparative Examples 1 to 2 were respectively taken by 2 mL (2 mL of each composition was removed), cast on an 8-inch wafer, spin-coated at a main speed of 1,500 rpm for 20 seconds by utilizing an auto track (ACT-8, TEL (Tokyo Electron Limited)), and cured at 210° C. for 90 seconds, forming 50 Å-thick thin layers.

Each thickness at 51 points along a horizontal axis was measured to evaluate coating uniformity, and the results are shown in Table 3.

TABLE 3

| | Coating uniformity (min/max range) |
|---|---|
| Example 1 | 1.8 Å |
| Example 2 | 1.3 Å |
| Example 3 | 1.2 Å |
| Example 4 | 2.0 Å |
| Example 5 | 1.4 Å |
| Example 6 | 1.0 Å |
| Example 7 | 2.8 Å |
| Comparative Example 1 | 7.3 Å |
| Comparative Example 2 | 5.7 Å |

Referring to Table 3, the resist underlayer compositions according to Examples 1 to 7 exhibited excellent or suitable coating uniformity, compared with the resist underlayer compositions according to the comparative examples.

### Evaluation of Film Density

The resist underlayer compositions according to Examples 1 to 7 and Comparative Examples 1 to 2 were respectively coated on a silicon substrate in a spin-on coating method, heat-treated at 210° C. on a hot plate for 90 seconds, forming 100 nm-thick resist underlayers.

Subsequently, the resist underlayers were measured with respect to density, and results are shown in Table 4. The density of the resist underlayers were measured by utilizing an X-ray diffractometer (Model: X'Pert PRO MPD, Malvern Panalytical Ltd. (Netherlands)).

TABLE 4

| | Film density (g/cm³) |
|---|---|
| Example 1 | 1.42 |
| Example 2 | 1.38 |
| Example 3 | 1.40 |
| Example 4 | 1.39 |
| Example 5 | 1.38 |
| Example 6 | 1.40 |
| Example 7 | 1.36 |
| Comparative Example 1 | 1.31 |
| Comparative Example 2 | 1.35 |

Referring to Table 4, the films formed of the resist underlayer compositions according to Examples 1 to 7 exhibited higher density than those formed of the resist underlayer compositions according to the comparative examples. It is believed that the film density was increased by including a polymer including a heterocycle containing two or more nitrogen atoms in the ring and a compound including a heterocyclic group and an aromatic hydrocarbon group.

Referring to the results of Table 4, the resist underlayer compositions according to Examples 1 to 7 was formed into films with a denser structure, compared with the films according to the comparative examples.

The use of “may” when describing embodiments of the present disclosure refers to “one or more embodiments of the present disclosure.”

As used herein, the term “substantially,” “about,” and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this disclosure is intended to include all higher numerical limitations subsumed therein. Accordingly, Applicant reserves the right to amend this disclosure, including the claims, to expressly recite any sub-range subsumed within the ranges expressly recited herein.

The photoresist coating apparatus, the metal-containing resist or any other relevant metal-containing resist manufacture, control or management devices or components according to embodiments of the present disclosure described herein may be implemented utilizing any suitable hardware, firmware (e.g., an application-specific integrated circuit), software, or a combination of software, firmware, and hardware. For example, the various components of the apparatus or device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Although the embodiments of the present disclosure have been described, it is understood that the present disclosure should not be limited to these embodiments, but one or more suitable changes and modifications can be made by one ordinary skilled in the art within the spirit and scope of the present disclosure as defined by the following claims and equivalents thereof.

### REFERENCE NUMERALS

100: substrate
102: thin layer
104: resist underlayer
106: photoresist layer
106a: exposed region of photoresist layer
106b: non-exposed region of photoresist layer
108: photoresist pattern
110: mask
112: organic layer pattern
114: thin layer pattern

What is claimed is:

1. A resist underlayer composition, the composition comprising:
a polymer comprising:
a main chain comprising a heterocycle comprising two or more nitrogen atoms in the ring of the heterocycle;
a side chain comprising a heterocycle comprising two or more nitrogen atoms in the ring of the heterocycle, or
a main chain and a side chain comprising a heterocycle comprising two or more nitrogen atoms in the ring of the heterocycle;
a compound comprising a moiety represented by Chemical Formula 1; and
a solvent: 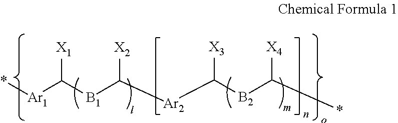
wherein, in Chemical Formula 1,
Ar₁ is a group comprising a heterocycle,
Ar₂ is a substituted or unsubstituted C6 to C30 aromatic hydrocarbon group,
B₁ and B₂ are each independently a single bond, a substituted or unsubstituted C1 to C10 aliphatic hydrocarbon group, a substituted or unsubstituted C6 to C30 aromatic hydrocarbon group, or a combination thereof,
X₁ to X₄ are each independently hydrogen, deuterium, a hydroxy group, a thiol group, a cyano group, a substituted or unsubstituted amino group, a halogen atom, a substituted or unsubstituted C1 to C30 aliphatic hydrocarbon group, a substituted or unsubstituted C1 to C30 alkoxy group, a substituted or unsubstituted C6 to C30 aromatic hydrocarbon group, a substituted or unsubstituted C2 to C30 heterocyclic group, or a combination thereof,
I, m, and n are each independently an integer from 0 to 5, and
o is an integer from 1 to 30.

2. The resist underlayer composition of claim 1, wherein
the polymer comprises one or more of the structural units represented by Chemical Formula 2 to Chemical Formula 5: 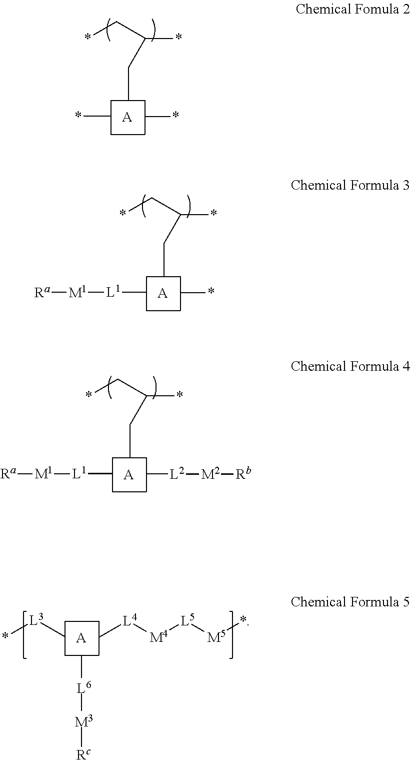
wherein, in Chemical Formula 2 to Chemical Formula 5,
A is a heterocycle containing two or more nitrogen atoms in the ring of the heterocycle,
R^{a}, R^{b}, and R^{c} are each independently a hydroxy group, a substituted or unsubstituted C1 to C10 alkyl group, a substituted or unsubstituted C2 to C10 alkenyl group, a substituted or unsubstituted C2 to C10 alkynyl group, a substituted or unsubstituted C1 to C10 heteroalkyl group, a substituted or unsubstituted C1 to C10 heteroalkenyl group, a substituted or unsubstituted C1 to C10 heteroalkynyl group, a substituted or unsubstituted C3 to C20 cycloalkyl group, a substituted or unsubstituted C2 to C20 heterocycloalkyl group, a substituted or unsubstituted C6 to C20 aryl group, a substituted or unsubstituted C1 to C20 heteroaryl group, or a combination thereof,
L¹ to L⁵ are each independently a single bond, a substituted or unsubstituted C1 to C10 alkylene group, a substituted or unsubstituted C1 to C10 heteroalkylene group, a substituted or unsubstituted C3 to C20 cycloalkylene group, a substituted or unsubstituted C2 to C20 heterocycloalkylene group, a substituted or unsubstituted C6 to C20 arylene group, a substituted or unsubstituted C1 to C20 heteroarylene group, or a combination thereof,
M¹ to M⁵ are each independently a single bond, —O—, —S—, —S(═O)—, —S(═O)₂—, —C(═O)—, —(CO)O—, —O(CO)O—, —NR″″— (wherein, R″″ is hydrogen, deuterium, or a C1 to C10 alkyl group), or a combination thereof, and
* is a linking point.

3. The resist underlayer composition of claim 2, wherein
A of Chemical Formula 2 to Chemical Formula 5 is represented by at least one of structures represented by Chemical Formula A-1 to Chemical Formula A-4: 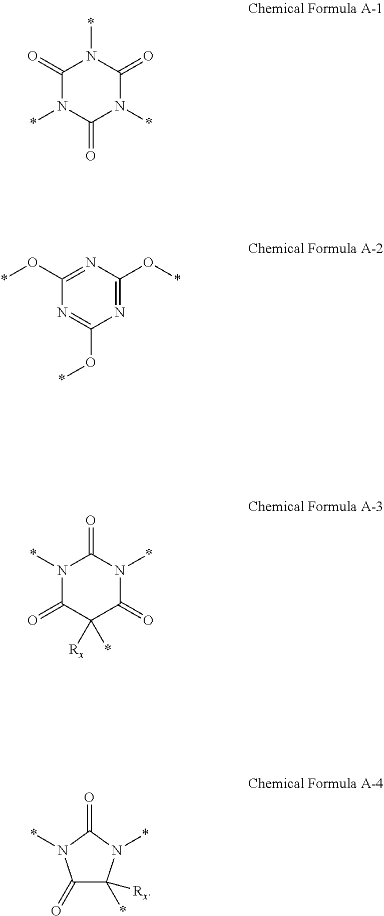
wherein, in Chemical Formula A-1 to Chemical Formula A-4,
Rₓ is each independently hydrogen, a substituted or unsubstituted C1 to C10 alkyl group, a substituted or unsubstituted C2 to C10 alkenyl group, a substituted or unsubstituted C2 to C10 alkynyl group, a substituted or unsubstituted C1 to C10 heteroalkyl group, a substituted or unsubstituted C1 to C10 heteroalkenyl group, a substituted or unsubstituted C1 to C10 heteroalkynyl group, a substituted or unsubstituted C3 to C20 cycloalkyl group, a substituted or unsubstituted C2 to C20 heterocycloalkyl group, a substituted or unsubstituted C6 to C20 aryl group, a substituted or unsubstituted C1 to C20 heteroaryl group, or a combination thereof, and
* is a linking point.

4. The resist underlayer composition of claim 1, wherein
Ar₁ of Chemical Formula 1 is a group comprising a substituted or unsubstituted heterocycle selected from Group 1: 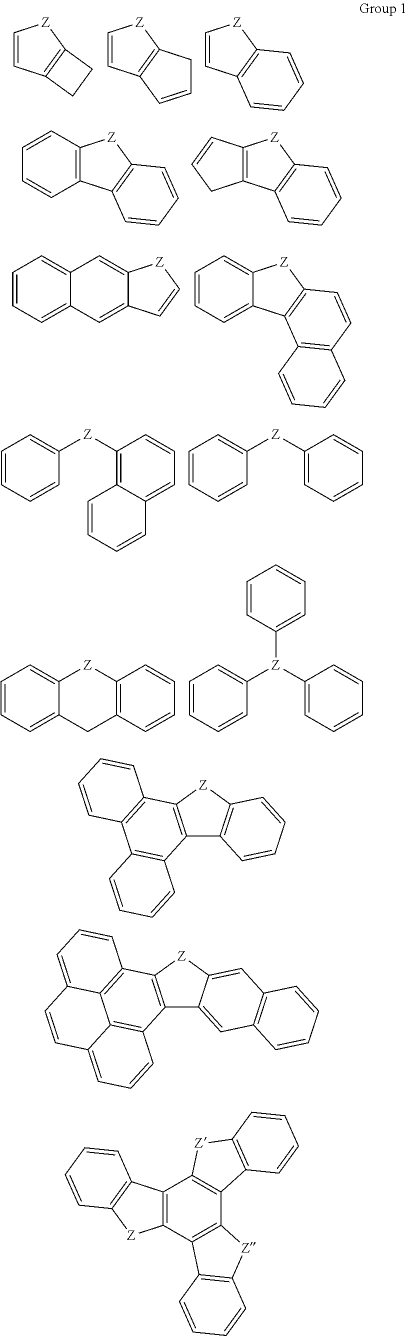 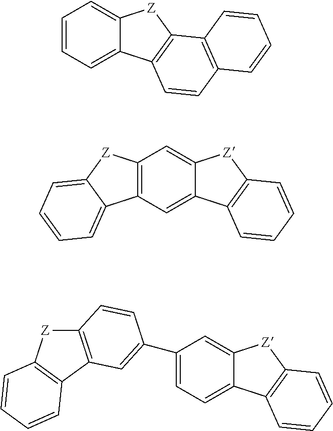 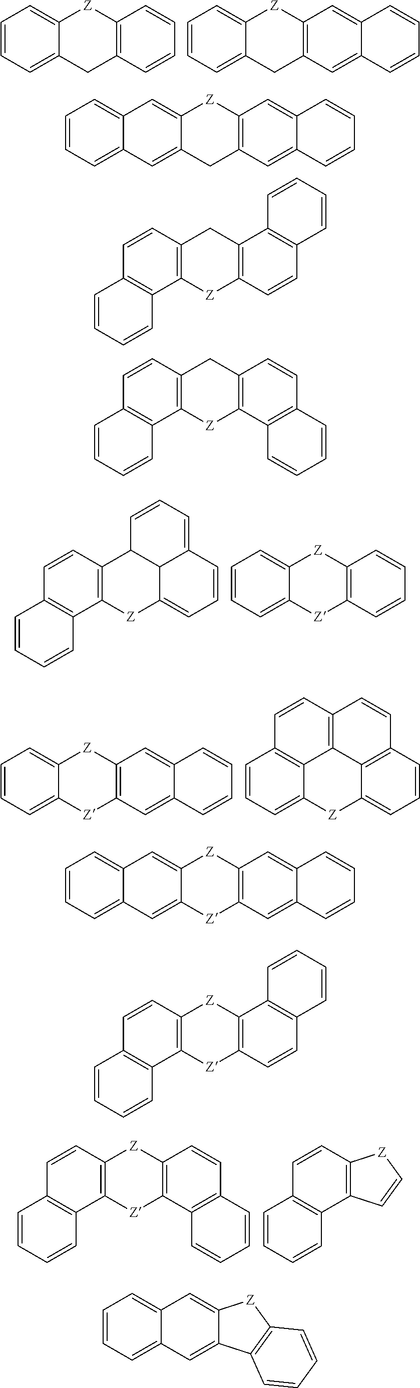 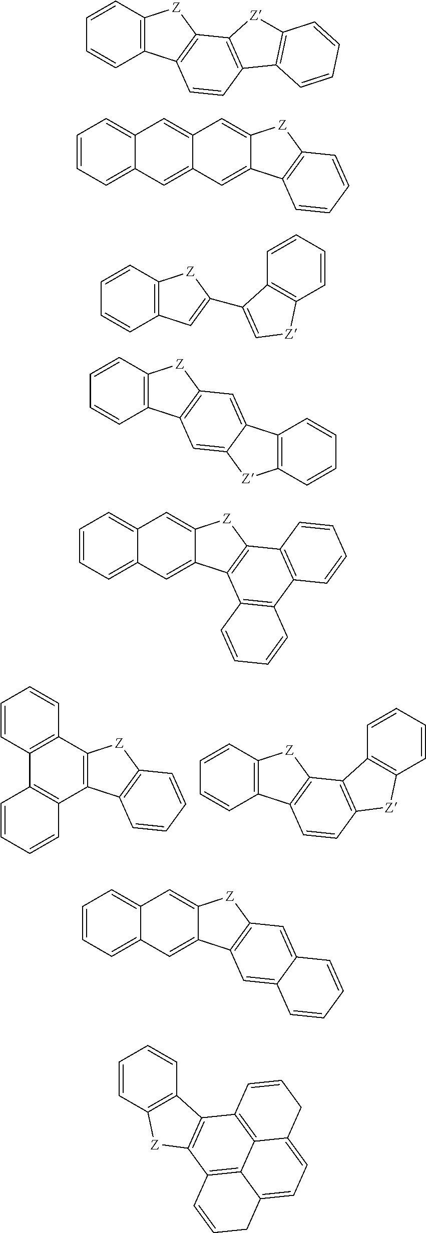 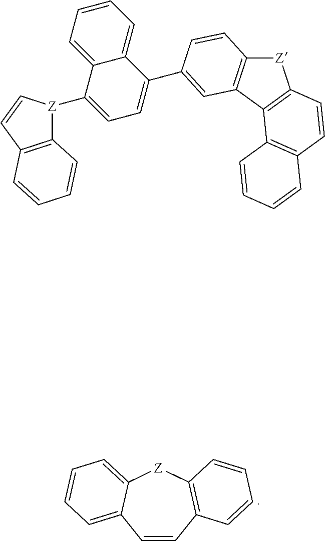
wherein, in Group 1,
Z, Z′, and Z″ are each independently N, O, S, or P, and
the substituted is replaced by a hydroxy group, a C1 to C10 alkoxy group, a cyano group, a halogen atom, a C1 to C30 alkyl group, a C2 to C30 alkenyl group, a C2 to C30 alkynyl group, a C3 to C30 cycloalkenyl group, NR′R″ (wherein R′ and R″ are each independently hydrogen, a C1 to C10 alkyl group, a C6 to C20 aryl group, or a combination thereof), a C1 to C20 heteroalkyl group, a C2 to C30 heterocycloalkyl group, a C2 to C30 heteroaryl group, or a combination thereof.

5. The resist underlayer composition of claim 1, wherein
Ar₂ of Chemical Formula 1 is a substituted or unsubstituted aromatic hydrocarbon group selected from Group 2: 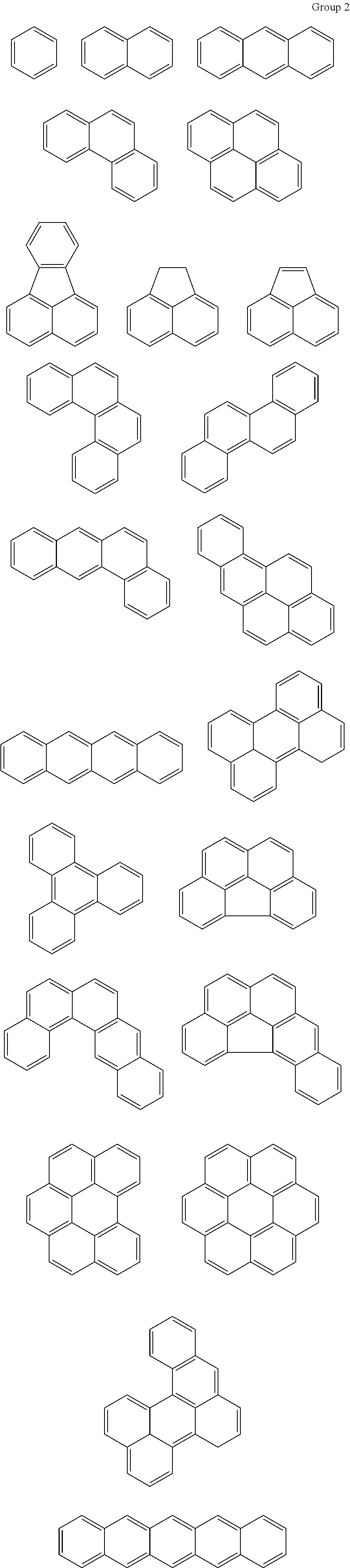
wherein, in Group 2,
the substituted is replaced by a hydroxy group, a C1 to C10 alkoxy group, a cyano group, a halogen atom, a C3 to C30 cycloalkenyl group, NR′R″(wherein R′ and R″ are each independently hydrogen, a C1 to C10 alkyl group, a C6 to C20 aryl group, or a combination thereof), a C1 to C20 heteroalkyl group, a C2 to C30 heterocycloalkyl group, a C2 to C30 heteroaryl group, or a combination thereof.

6. The resist underlayer composition of claim 1, wherein
Ar₁ of Chemical Formula 1 is a group comprising a substituted or unsubstituted heterocycle selected from Group 1-1:

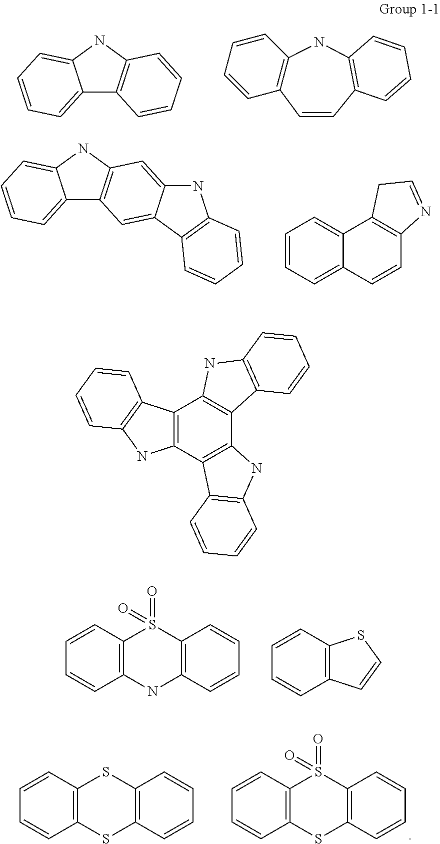

7. The resist underlayer composition of claim 6, wherein
the substituted is replaced by a hydroxy group, a C1 to C10 alkoxy group, a cyano group, a halogen atom, a C1 to C30 alkyl group, a C2 to C30 alkenyl group, a C2 to C30 alkynyl group, a C3 to C30 cycloalkenyl group, NR′R″(wherein R′ and R″ are each independently hydrogen, a C1 to C10 alkyl group, a C6 to C20 aryl group, or a combination thereof), a C1 to C20 heteroalkyl group, a C2 to C30 heterocycloalkyl group, a C2 to C30 heteroaryl group, or a combination thereof.

8. The resist underlayer composition of claim 1, wherein
Ar₂ of Chemical Formula 1 is a substituted or unsubstituted aromatic hydrocarbon group selected from Group 2-1:

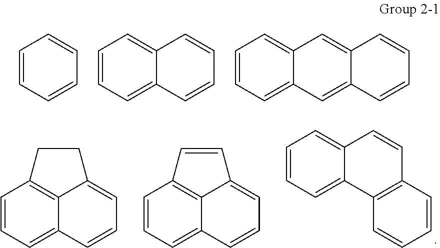

9. The resist underlayer composition of claim 8, wherein
the substituted is replaced by a hydroxy group, a C1 to C10 alkoxy group, a cyano group, a halogen atom, a C3 to C30 cycloalkenyl group, NR′R″ (wherein, R′ and R″ are each independently hydrogen, a C1 to C10 alkyl group, a C6 to C20 aryl group, or a combination thereof), a C1 to C20 heteroalkyl group, a C2 to C30 heterocycloalkyl group, a C2 to C30 heteroaryl group, or a combination thereof.

10. The resist underlayer composition of claim 1, wherein
B₁ and B₂ of Chemical Formula 1 each independently comprise a substituted or unsubstituted one selected from Group 3:

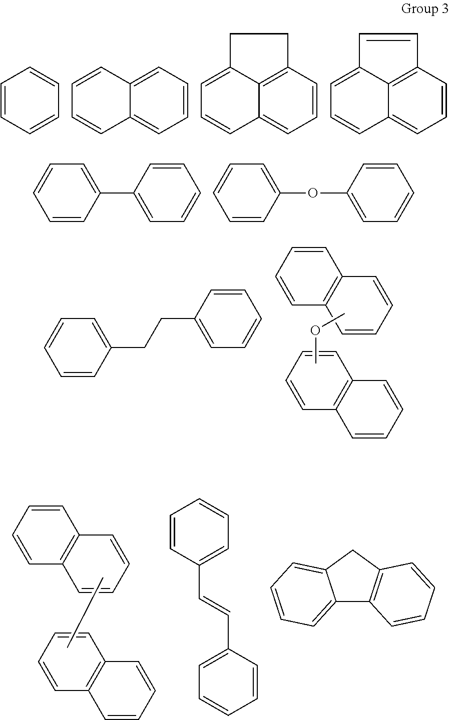

11. The resist underlayer composition of claim 10, wherein
the substituted is replaced by a hydroxy group, a C1 to C10 alkoxy group, a cyano group, a halogen atom, a C3 to C30 cycloalkenyl group, NR′R″(wherein R′ and R″ are each independently hydrogen, a C1 to C10 alkyl group, a C6 to C20 aryl group, or a combination thereof.), a C1 to C20 heteroalkyl group, a C2 to C30 heterocycloalkyl group, a C2 to C30 heteroaryl group, or a combination thereof.

12. The resist underlayer composition of claim 1, wherein
a weight ratio of the polymer to the compound is about 9:1 to about 1:9.

13. The resist underlayer composition of claim 1, wherein
a weight average molecular weight of the polymer is about 2,000 g/mol to about 300,000 g/mol.

14. The resist underlayer composition of claim 1, wherein
a molecular weight of the compound is about 300 g/mol to about 5,000 g/mol.

15. The resist underlayer composition of claim 1, wherein
a molecular weight of the compound is about 1,000 g/mol to about 50,000 g/mol.

16. The resist underlayer composition of claim 1, wherein
the resist underlayer composition further comprises one or more polymers selected from among an acrylic resin, an epoxy resin, a novolac resin, a glycoluril-based resin, and a melamine-based resin.

17. The resist underlayer composition of claim 1, wherein
the resist underlayer composition further comprises an additive comprising a crosslinking agent, a thermal acid generator, a surfactant, a plasticizer, or a combination thereof.

18. A method of forming patterns, the method comprising:
forming an etching-objective layer on a substrate;
coating the resist underlayer composition of claim 1 on the etching-objective layer;
forming a resist underlayer from the resist underlayer composition;
forming a photoresist pattern on the resist underlayer; and
sequentially etching the resist underlayer and the etching-objective layer utilizing the photoresist pattern as an etch mask.

19. The method of claim 18, wherein
the forming of the resist underlayer comprises heat-treating the resist underlayer composition at a temperature of about 100° C. to about 500° C. after the coating of the resist underlayer composition.

20. The method of claim 18, wherein
the forming of the photoresist pattern comprises:
forming a photoresist layer on the resist underlayer;
exposing the photoresist layer; and
developing the photoresist layer.
